(19) United States
(12) Patent Application Publication
(10) Pub. No.: US 2021/0187040 A1
(43) Pub. Date: Jun. 24, 2021

(54) IPSC-DERIVED SECRETOME COMPOSITIONS, AND RELATED SYSTEMS AND METHODS

(71) Applicant: Orig3n, Inc., Boston, MA (US)

(72) Inventors: Robin Y. Smith, Boston, MA (US); Marcie A. Glicksman, Boston, MA (US)

(21) Appl. No.: 16/643,812

(22) PCT Filed: Mar. 14, 2018
(86) PCT No.: PCT/US18/22325

§ 371 Date: Mar. 2, 2020

Related U.S. Application Data
(60) Provisional application No. 62/595,447, filed on Dec. 6, 2017.
(60) Provisional application No. 62/592,263, filed on Nov. 29, 2017.
(60) Provisional application No. 62/553,545, filed on Sep. 1, 2017.

(51) Int. Cl. A61K 35/545; C12N 5/077; C12N 5/074; C07K 14/52; C12N 15/113; C12N 9/22; C12N 5/078; C12N 5/0775

(52) U.S. Cl. CPC A61K 35/545; C12N 5/0655; C12N 5/0696; C07K 14/52; A61K 38/00; C12N 9/22; C12N 5/0634; C12N 5/0668; C12N 15/113

(57) ABSTRACT

Presented herein are methods of producing “personalized” secretome compositions suitable for secretome based therapy to be administered to a specific individual and/or specific group of individuals. The iPSCs and/or iPSC-derived cells, and any iPSC-derived compositions derived therefrom, are identified as compatible with a specific individual or specific group of individuals using an identification of a cell type indicative of compatibility such as an HLA match and/or ABO blood match and/or RHD blood group match. The identified compatible cells are then retrieved from a managed HLA-indexed (and/or otherwise indexed) repository or are derived from a biological sample of a suitable donor. The retrieved compatible cells are then used to derive the “personalized” iPSC-derived secretome compositions, that comprise the complete secretome or a subset of the secretome suitable for treatment of a specific individual and/or specific group of individuals.

### CROSS REFERENCE TO RELATED APPLICATIONS

This application claims the benefit of U.S. Provisional Application No. 62/553,545 filed Sep. 1, 2017, U.S. Provisional Application No. 62/592,263 filed Nov. 29, 2017, and U.S. Provisional Application No. 62/595,447 filed Dec. 6, 2017, the contents of which are hereby incorporated by reference herein in their entirety.

### TECHNICAL FIELD

The invention relates generally to apparatus, systems, and methods related to secretome compositions derived from induced pluripotent stem cells (iPSCs), and related systems and methods.

### BACKGROUND

The secretome refers to the totality of organic and inorganic elements and molecules secreted by a cell, tissue, organ, or organism into its environment. This includes but is not limited to secreted proteins, microvesicles, and exosomes. While various proteins are secreted by the cell into its environment, cytokines are of special interest. “Cytokines” is a general name for a class of small intercellular proteins secreted by specific cells to mediate and regulate the immune response, inflammation, and hematopoiesis in the human body. They are broadly divided into pro-inflammatory cytokines and anti-inflammatory cytokines, with the latter keeping in check the former's response. Anti-inflammatory cytokines can be used to prevent or attenuate hyperalgesia and allodynia, for skin rejuvenation and treatment, damaged organ treatment and also for disease treatment.

Microvesicles and exosomes are cellular structures secreted by the cell. Microvesicles refer to the small circular fragments of plasma membrane shed by almost all types of cells. Alternatively, exosomes are smaller vesicles generated intracellularly by the cells and then secreted out of the cell. Both microvesicles and exosomes play a key role in cell-to-cell communication and are used to transport mRNA, miRNA, siRNA, and proteins between cells.

Where allogeneic cells are needed, a suitable donor (someone other than the patient) must be found for the patient in order to minimize risk of rejection and maximize chances for success. Donor registries are services that seek to match registered donors with patients in need of an allogeneic transplant. Matching based on human leukocyte antigen (HLA) typing is typically performed to find suitable donors. Because there are many different HLA types, it is often difficult to find suitable matches, particularly when no family members of the patient are an HLA-identical match.

The term “super donors” refers to human leukocyte antigen (HLA) types (or cell lines or individuals having those HLA types) that do not trigger strong rejection reactions. Super donors have a HLA haplotype that is common among the population and will match a sizable portion of a particular population. This is analogous to banking a blood transfusion from a donor who has blood type O-negative, which can be tolerated by patients of all blood types.

Humans are almost always heterozygous for a particular HLA gene—that is, genotyping data shows that humans usually express two different alleles. For a successful match, eight (8) HLA alleles are best for matching (4 alleles on each of the donor and recipients chromosomes). With homozygous donors, only 4 alleles are required to be matched, therefore increasing the number of recipients that would be a match to the donor. Individuals that are homozygous for all three key HLA alleles that govern rejection means that only three genes need to be genotyped and matched instead of six genes. iPSCs function like embryonic stem cells in that they can be differentiated into a variety of different cell types. iPSC lines derived from these so-called “super donors” can be used to reduce immunogenicity. It is believed that about 200 such iPSC lines could cover a high percentage (e.g., at least 90%, at least 95%, or more) of the U.S. and/or European population, and about 90 to 100 such iPSC lines could cover a high percentage (e.g., at least 90%, at least 95%, or more) of the Japanese population.

Recently, cytokines and secretomes have been successfully produced from iPSCs and also used for treatment of various cosmetic conditions and diseases. See, for example, “Exosomes Generated From iPSC-Derivatives New Direction for Stem Cell Therapy in Human Heart Diseases”, Cir. Res. 2017 January; 120(2): 407-417; “The secretome of induced pluripotent stem cells reduces lung fibrosis in part by hepatocyte growth factor”, Stem Cell Res. Ther. 2014 November; 5(123): 1-11; “Exosomes secreted by human-induced pluripotent stem cell-derived mesenchymal stem cells attenuate limb ischemia by promoting angiogenesis in mice”, Stem Cell Res. Ther. 2015 April; 6(10): 1-15; “Induced pluripotent stem cell (iPSCs) and their application in immunotherapy”, Cell Mol. Immunol. 2014 January; 11(1): 17-24; “Human growth factor and cytokine skin cream for facial skin rejuvenation as assessed by 3D in vivo optical skin imaging”, J. Drugs Dermatol. 2007 October; 6(10): 1018-23; “Skin rejuvenation using cosmetic products containing growth factors, cytokines, and matrikines: a review of the literature,” J. Drugs Dermatol., 2007 February; 6(2): 197-200; and “Anti-cytokine therapy for Rheumatoid Arthritis,” Blood, 2000 February; 51: 207-29; the contents of each of which are incorporated herein by reference. Furthermore, in recent years, there have been significant advances in the production of iPSCs from cells collected from a biological sample of a subject (e.g., blood cells). For example, iPSCs can be made by inserting copies of stem cell-associated genes—e.g., Oct 3/4, Sox 2, Klf4, and c-Myc (or Oct 3/4, Sox 2, Nanog, and Lin28)—into cells collected from the biological sample using viral vectors. See, for example, K. Okita, T. Ichisaka, and S. Yamanaka, “Generation of germline-competent induced pluripotent stem cells,” Nature, vol. 448, no. 7151, pp. 313-317, 2007; K. Okita, Y. Matsumura, Y. Sato et al., “A more efficient method to generate integration-free human iPS cells,” Nature Methods, vol. 8, no. 5, pp. 409-412, 2011; the contents of each of which are incorporate herein by reference.

There is a need for more effective compositions for secretome and cytokine therapy and advances in methods of producing them.

### SUMMARY

Presented herein are methods of producing “personalized” secretome compositions suitable for secretome based therapy (e.g., suitable for cytokine therapy and/or exosome therapy and/or microvesicle therapy) to be administered to a specific individual and/or specific group of individuals. Reserves of induced pluripotent stem cells (iPSCs) and other iPSC-derived cells (e.g., hematopoietic stem cell (HSCs), blood progenitor cells, Retinal Pigment Epithelium (RPE), chondrocytes, mesenchymal stem cells (MSCs), embryoid bodies and the like), iPSC lines and other iPSC-derived cell lines (e.g., HSC lines, blood progenitor cell lines, MSC lines, REP lines, and the like), and secretomes derived from these cells and/or cell lines are stored in a managed physical repository (e.g., a bank) for providing a resource (e.g., donors for secretome treatment therapy) for patients. This managed repository of cells, and/or cell lines, and/or secretomes derived from iPSCs (or embryoid bodies formed from iPSCs), also stores corresponding data comprising a set of characterized HLA loci, said corresponding data being stored in a searchable database for retrieval of one or more matching physical cell lines upon query. The repository comprises a bank of cells (e.g., iPSCs, embryoid bodies, HSCs, MSCs, RPEs, blood progenitor cells and/or various other cells) derived from iPSCs, cell lines (HSCs, MSCs, RPEs, blood progenitor cells and/or various other cell lines derived from iPSCs), along with secretomes derived from each of these cells and/or cell lines (E.g., iPSC-derived secretomes), for each of a set of HLA types. This repository of cells, and/or cell lines and/or iPSC-derived secretomes allows for identification and provision of allogenic cell lines and iPSC-derived secretomes suitable for transplantation and/or treatment to reestablish normal function in patients with various diseases and/or conditions.

The techniques described herein allow for the tuning of secretome compositions to a specific individual or a specific group of individuals, thus enabling improved methods of secretome based therapy, e.g. due to an enhanced compatibility of the specific individual or group of individuals with the cells from which the desired secretome composition is derived. Also, allogeneic iPS cells and/or cell lines that are compatible with a large portion of a specific population, e.g. super donors, can be prepared and stored in advance for large groups of individuals. These super donor-derived secretome compositions can then be made immediately available to people who need them, thus reducing production times of the iPSC-derived secretome compositions.

In one aspect, the invention is directed to a method of manufacturing an induced pluripotent stem cell (iPSC)-derived secretome composition tailored for treatment of a particular subject or particular group of subjects, said method comprising the steps of: (a) identifying, as compatible with the particular subject or particular group of subjects, one or more iPSCs and/or one or more iPSC-derived cells; (b) retrieving compatible cells corresponding to the one or more iPSCs and/or one or more iPSC-derived cells identified as compatible with the particular subject or particular group of subjects; and (c) producing the iPSC-derived secretome composition using the retrieved compatible cells.

In certain embodiments, the one or more iPSCs and/or the one or more iPSC-derived cells are human cells (e.g., in certain other embodiments, the one or more iPSCs and/or the one or more iPSC-derived cells are non-human animal cells).

In certain embodiments, the iPSC-derived secretome composition comprises one or more desired compatible-cell-secreted species.

In certain embodiments, the one or more desired compatible-cell-secreted species comprise one or more desired compatible-cell-secreted molecules and/or one or more desired compatible-cell-secreted biological elements. In certain embodiments, the one or more desired compatible-cell-secreted species comprise one or more cytokines. In certain embodiments, the one or more desired compatible-cell-secreted species comprise one or more exosomes and/or one or more microvesicles.

In certain embodiments, step (c) comprises extracting one or more desired compatible-cell-secreted molecules and/or one or more desired biological elements from the retrieved compatible cells.

In certain embodiments, step (b) comprises deriving the compatible cells from a biological sample of the particular subject.

In certain embodiments, step (c) comprises producing a lyophilized iPSC-derived secretome composition.

In certain embodiments, the retrieved compatible cells comprise one or more members selected from the group consisting of induced pluripotent stem cells (iPSCs), mesenchymal stem cells (MSCs), Retinal Pigment Epithelium (RPEs), chondrocytes, hematopoietic stem cells (HSCs), blood progenitor cells, and embryoid bodies.

In certain embodiments, the particular subject or the particular group of subjects is/are human.

In certain embodiments, the one or more iPSCs and/or one or more iPSC-derived cells are stored in a physical repository.

In certain embodiments, step (b) comprises obtaining the compatible cells from a physical repository.

In certain embodiments, step (b) comprises retrieving, by a processor of a computing device, one or more data entries corresponding to the compatible cells using a processor-based query from a user, wherein the query comprises an identification of a cell type indicative of compatibility with the particular subject or particular group of subjects.

In certain embodiments, the identification of cell type indicative of compatibility with the particular subject or particular group of subjects comprises one or more of (i) to (iii): (i) an HLA match, (ii) an ABO blood type match, and (iii) an RHD blood group match.

In certain embodiments, the iPSC-derived secretome composition comprises the retrieved compatible cells.

In certain embodiments, step (c) comprises forming the retrieved compatible cells into a macroscopic structure suitable for topical application to the subject. In certain embodiments, the macroscopic structure is a sheet.

In certain embodiments, producing the iPSC-derived secretome composition in step (c) comprises exposing the compatible cells to culture media.

In certain embodiments, the iPSC-derived secretome composition comprises the compatible cells, the culture media, and the one or more desired compatible-cell-secreted species.

In certain embodiments, step (c) comprises producing blood progenitor cells and/or HSCs and/or MSCs and/or embryoid bodies and/or RPEs and/or chondrocytes from the one or more iPSCs identified as compatible with the particular subject or particular group of subjects.

In certain embodiments, the method comprises producing the iPSC-derived secretome composition from the produced blood progenitor cells, and/or produced HSCs, and/or produced MSCs, and/or produced embryoid bodies, and/or produced RPEs, and/or produced chondrocytes.

In certain embodiments, the iPSC-derived secretome composition is a treatment spray, or a treatment cream, or a lotion. In certain embodiments, the iPSC-derived secretome composition is a treatment injection.

In another aspect, the invention is directed to a method of manufacturing an induced pluripotent stem cell (iPSC)-derived secretome composition tailored for treatment of a particular subject or particular group of subjects, said method comprising the steps of: (a) storing, by a processor of a computing device, a database comprising a data entry corresponding to each of a plurality of characterized cells in a physical repository, wherein the characterized cells comprise iPSCs and/or iPSC-derived cells; (b) receiving, by the processor, a query from a user comprising an identification of a cell type (e.g., HLA type) of the particular subject or particular group of subjects; (c) matching, by the processor, the query to one or more data entries of the database, each of the matching data entries corresponding to each of the plurality of characterized cells having a cell type compatible with the particular subject or particular group of subjects, thereby identifying as compatible with the subject the one or more characterized cells; (d) retrieving, from a physical repository, compatible cells corresponding to the one or more characterized cells identified as compatible with the particular subject or particular group of subjects; and (e) producing the iPSC-derived secretome composition using the retrieved compatible cells.

In certain embodiments, the data entry corresponding to each of the plurality of characterized cells comprises a set of characterized HLA loci corresponding to the cell, the query comprises a set of queried HLA loci for the particular subject or the particular group of subjects, and the one or more matched data entries of the database are each representative of one or more characterized compatible cells matching the queried HLA loci.

In certain embodiments, the plurality of characterized cells in the physical repository are immortalized.

In certain embodiments, the set of characterized HLA loci corresponding to each of the plurality of characterized cells comprises a set of at least 3 HLA loci, wherein the at least 3 HLA loci are HLA-A, HLA-B, and HLA-DRB.

In certain embodiments, the set of characterized HLA loci corresponding to each of the plurality of characterized cells comprises a set of at least 9 given loci, wherein the at least 9 given loci are HLA-A, HLA-B, HLA-C, HLA-DRB1, HLA-DRB3, HLA-DRB4, HLA-DRB5, HLA-DQB1, HLA-DPB1.

In certain embodiments, the set of characterized HLA loci corresponding to each of the plurality of characterized cells comprises at least 3 (e.g., at least 3, at least 4, at least 5, at least 6, at least 7, at least 8, or at least 9 members are selected from the at least 9 given loci) given loci selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, each of the one or more matching data entries of the database exactly match or partially match the set of queried HLA loci for the particular subject or the particular group of subjects.

In certain embodiments, the data entry for each of the plurality of characterized cells further comprises ABO blood type and the query further comprises ABO blood type, and wherein the one or more matching data entries of the database representative of the one or more characterized compatible cells match the queried HLA loci and the queried ABO blood type.

In certain embodiments, the data entry for each of the plurality of characterized cells further comprises RHD blood group and the query further comprises RHD blood group, and wherein the one or more matching data entries of the database representative of the one or more characterized compatible cells match the queried RHD blood group and the queried HLA loci.

In certain embodiments, the queried HLA loci correspond to the particular subject or particular group of subjects in need of an HLA matched iPSC-derived secretome composition.

In certain embodiments, the HLA matched iPSC-derived secretome composition is selected from one or more iPSC-derived secretome compositions, each derived from the one or more characterized compatible cells corresponding to each of the one or more data entries of the database that exactly match or partially match the queried HLA loci of the particular subject.

In certain embodiments, one or more of the queried HLA loci is determined by processing and analyzing a biological sample from the particular subject in need of the HLA match.

In certain embodiments, the queried ABO blood type is determined by processing and analyzing a biological sample from the particular subject in need of an ABO match.

In certain embodiments, the queried RHD blood group is determined by processing and analyzing a biological sample from the particular subject in need of a RHD blood group match.

In certain embodiments, the physical repository comprises one or more liquid nitrogen storage tanks (e.g., and/or another freezer system).

In certain embodiments, the method comprises producing blood progenitor cells and/or HSCs and/or MSCs and/or RPEs and/or chondrocytes from each of the one or more characterized compatible cells corresponding to the one or more data entries matching the queried HLA loci.

In certain embodiments, the method further comprises administering the iPSC-derived secretome composition to the particular subject or particular group of subjects. In certain embodiments, the administering step comprises administering the iPSC-derived secretome composition to the particular subject or particular group of subjects for treatment of a known disease, injury, or condition in the particular subject or particular group of subjects, wherein the known disease, injury, or condition is a member selected from the group consisting of lung disease, rheumatic diseases, cardiovascular disease, cancer, arthritis, traumatic brain injury, central nervous system (CNS) injury, and inflammation.

In certain embodiments, the database comprises a data entry corresponding to each of a plurality of iPSC super donor cell lines, wherein the data entry for each super donor cell line comprises a set of characterized HLA loci corresponding to the super donor cell line.

In certain embodiments, each of the plurality of iPSC super donor cell lines can be used for treatment of a particular subject or particular group of subjects having matching HLA loci with lower risk of immune rejection by the particular subject or particular group of subjects.

In certain embodiments, the method further comprises determining the set of characterized HLA loci corresponding to each of the plurality of super donor cell lines by processing and analyzing one or more biological samples collected from each of one or more super donor individuals.

In certain embodiments, the step of determining the set of characterized HLA loci corresponding to each of the plurality of super donor cell lines comprises identifying a set of at least 3 HLA loci, wherein the at least 3 HLA loci are HLA-A, HLA-B, and HLA-DRB.

In certain embodiments, the step of determining the set of characterized HLA loci corresponding to each of the plurality of the super donor cell lines comprises identifying a set of at least 9 HLA loci, wherein the at least 9 HLA loci are HLA-A, HLA-B, HLA-C, HLA-DRB1, HLA-DRB3, HLA-DRB4, HLA-DRB5, HLA-DQB1, and HLA-DPB1.

In certain embodiments, the set of characterized HLA loci corresponding to each of the plurality of the super donor cell lines comprises at least 3 (e.g., at least 4, at least 5, at least 6, at least 7, at least 8, or at least 9) HLA loci selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, the set of characterized HLA loci corresponding to each of the plurality of the super donor cell lines are homozygous for HLA-A, HLA-B, and DRB-1.

In certain embodiments, the homozygous set of characterized HLA loci belong to a set of most-common HLA loci for a given population that matches a majority of the given population.

In certain embodiments, the homozygous set of characterized HLA loci comprise homozygous HLA loci in at least 3 major sites (e.g., or at least 4, or at least 5, or at least 6, or at least 7, or at least 8, or at least 9 major sites) wherein the major sites are members selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, the plurality of iPSC super donor cell lines match at least 70% (e.g., at least 75%, at least 80%, at least 85%, at least 90%, or at least 95%) of the population from which the particular subject originates.

In certain embodiments, the iPSC-derived secretome composition is produced using one of the plurality of iPSC super donor cell lines.

In certain embodiments, the method comprises exposing the iPSC super donor cell line used to produce the iPSC-derived secretome composition to culture media.

In certain embodiments, the iPSC-derived secretome composition comprises cells from the iPSC super donor cell line, the culture media, and one or more desired compatible-cell-secreted species. In certain embodiments, the one or more desired compatible-cell-secreted species comprise one or more desired compatible-cell-secreted molecules and/or one or more desired compatible-cell-secreted biological elements. In certain embodiments, the one or more desired compatible-cell-secreted species comprise one or more exosomes and/or one or more microvesicles.

In certain embodiments, the method comprises producing blood progenitor cells and/or HSCs and/or MSCs and/or RPEs and/or chondrocytes from each of one or more iPSC super donor cell lines identified as compatible with the particular subject or particular group of subjects.

In certain embodiments, the iPSC-derived secretome composition is a treatment spray. In certain embodiments, the iPSC-derived secretome composition is a treatment lotion or a treatment cream.

In certain embodiments, the iPSC-derived secretome composition comprises one or more proteins listed in Table 1, and/or Table 2, and/or Table 3, and/or Table 4.

In certain embodiments, the iPSC-derived secretome composition is for internal use. In certain embodiments, the iPSC-derived secretome composition is an injection. In certain embodiments, the iPSC-derived secretome composition is lyophilized.

In certain embodiments, the method comprises engineering the compatible cells to upregulate production of one or more desired proteins in the iPSC-derived secretome composition. In certain embodiments, the compatible cells are engineered using CRISPR/Cas9 technology. In certain embodiments, the method comprises removing and/or replacing and/or editing one or more genes of the compatible cells so as to increase the likelihood of the upregulation of one or more desired proteins in the iPSC-derived secretome composition.

In another aspect, the invention is directed to a composition of matter comprising an iPSC-derived secretome composition comprising one or more desired compatible-cell-secreted species, wherein the composition is produced by the method of any one of the aspects and embodiments described herein.

In certain embodiments, the iPSC-derived secretome composition is a member selected from the group consisting of a treatment spray, a treatment cream, a treatment lotion, and a treatment injection.

In certain embodiments, the iPSC-derived secretome composition comprises compatible cells, conditioned culture media, and one or more of the desired compatible-cell-secreted species.

In certain embodiments, the iPSC-derived secretome composition comprises one or more additives. In certain embodiments, the one or more additives comprises one or more nutrients and/or one or more supplements.

In certain embodiments, the iPSC-derived secretome composition comprises iPS cells that are derived from a biological sample of a particular subject.

In certain embodiments, the iPSC-derived secretome composition comprises compatible cells retrieved from a physical repository, wherein the compatible cells are identified as compatible with the particular subject or a particular group of subjects. In certain embodiments, the compatible cells are identified as compatible with the particular subject or the particular group of subjects using an identification of cell type indicative of compatibility with the particular subject or particular group of subjects, wherein the identification of cell type indicative of compatibility comprises one or more of (i) to (iii): (i) an HLA match, (ii) an ABO blood type match, and (iii) an RHD blood group match having the same HLA loci, and/or ABO blood type, and/or RHD blood group as the.

In certain embodiments, the iPSC-derived secretome composition comprises one or more compatible-cell-secreted species. In certain embodiments, the one or more compatible-cell-secreted species are one or more members selected from the group consisting of cytokines, miRNA, siRNA, proteins, organic molecules, inorganic molecules, and biological elements.

In certain embodiments, the iPSC-derived secretome composition comprises one or more proteins listed in Table 1, and/or Table 2, and/or Table 3, and/or Table 4.

In certain embodiments, the iPSC-derived secretome composition is formulated internal use. In certain embodiments, the iPSC derived secretome composition is formulated for use in an injection. In certain embodiments, the iPSC-derived secretome composition is lyophilized.

In certain embodiments, the iPSC-derived secretome composition comprises engineered compatible cells. In certain embodiments, the engineered compatible cells are modified to upregulate and/or downregulate production of one or more desired proteins in the iPSC-derived secretome composition. In certain embodiments, the engineered compatible cells are modified using CRISPR/Cas9 technology.

In another aspect, the invention is directed to a method of storing an induced pluripotent stem cell (iPSC)-derived secretome composition tailored for treatment of a particular subject or particular group of subjects, said method comprising the steps of: (a) identifying, by a processor of a computing device, as compatible with the particular subject or particular group of subjects, one or more iPSC-derived secretome compositions derived using compatible cells corresponding to the one or more iPSCs and/or iPSC-derived cells identified as compatible with the particular subject or particular group of subjects; (b) labelling, by a processor of a computing device, the one or more iPSC-derived secretome compositions with a label, wherein the label comprises information relating to the iPSCs and/or iPSC-derived cells, and a classification of the iPSC and/or IPSC-derived cells the iPSC-derived secretome composition is derived from; and (c) storing, by a processor of a computing device, a database comprising a data entry corresponding to each label in a physical repository.

In certain embodiments, the label is a physical label and/or a digital label.

In certain embodiments, the label comprises information relating to one or more of (i) to (iii) as follows: (i) the iPSCs and/or iPSC-derived cells the iPSC-derived secretome composition is derived from; (ii) one or more HLA loci, and/or ABO blood type, and/or RHD blood group compatible with the labeled iPSC-derived secretome composition; and (iii) one or more other iPSC-derived secretome compositions stored in the physical repository that are compatible with the particular subject or particular group of subjects, wherein the HLA loci, and/or ABO blood type, and/or RHD blood group of the one or more other iPSC-derived secretome compositions are identical to or match the HLA loci, and/or ABO blood type, and/or RHD blood group of the iPSCs and/or iPSC-derived cells of (i).

In another aspect, the invention is directed to a method of retrieving one or more produced, labeled and stored iPSC-derived secretome compositions derived using iPSCs and/or iPSC-derived cells, said method comprising the steps of: (a) identifying, by a processor of a computing device, as compatible with a particular subject or particular group of subjects, one or more iPSC-derived secretome compositions derived using one or more iPSCs and/or iPSC-derived cells identified as compatible with the particular subject or particular group of subjects; (b) retrieving from a physical repository the one or more compatible iPSC-derived secretome compositions corresponding to the one or more iPSCs and/or iPSC-derived cells identified as compatible with the particular subject or particular group of subjects; and (c) updating, by a processor of a computing device, a database comprising data entries corresponding to the particular subject or particular group of subjects.

In certain embodiments, the retrieved one or more iPSC-derived secretome compositions is administered as treatment to the subject. In certain embodiments, the treatment is a spray. In certain embodiments, the treatment is a cream and/or lotion. In certain embodiments, the treatment is an injection.

In another aspect, the invention is directed to a method of administering an iPSC-derived secretome composition tailored for treatment of a particular subject or particular group of subjects, said method comprising the steps of: (a) identifying the particular subject or particular group of subjects as having a deficiency in one or more substances; (b) identifying, as compatible with the particular subject or particular group of subjects, one or more iPSCs and/or one or more iPSC-derived cells; (c) retrieving compatible cells corresponding to the one or more iPSCs and/or one or more iPSC-derived cells identified as compatible with the particular subject or particular group of subjects; (d) producing the iPSC-derived secretome composition using the retrieved compatible cells, wherein the iPSC-derived secretome composition comprises the one or more substances deficient in the particular subject or the particular group of subjects; and (e) administering to the particular subject or particular group of subjects the iPSC-derived secretome composition.

In certain embodiments, the one or more substances comprise one or more cell-secreted molecules and/or cell-secreted biological elements.

In certain embodiments, the iPSC-derived secretome composition comprises the one or more cell-secreted substances identified to be deficient in the particular subject or the particular group of subjects.

In certain embodiments, step (d) comprises extracting the secretomes of the retrieved compatible cells.

In certain embodiments, step (c) comprises obtaining the compatible cells from a physical repository.

In certain embodiments, the compatible cells are one or more members selected from the group consisting of iPSCs, MSCs, RPEs, chondrocytes, embryoid bodies, HSCs, and blood progenitor cells.

In certain embodiments, step (c) comprises retrieving the compatible cells using a processor-based query from a user, wherein the query comprises an identification of a cell type indicative of compatibility with the particular subject or particular group of subjects.

In certain embodiments, the identification of cell type indicative of compatibility with the particular subject or particular group of subjects comprises one or more of (i) to (iii): (i) an HLA match, (ii) an ABO blood type match, and (iii) an RHD blood group match.

In certain embodiments, step (b) comprises identifying, one or more stored and labeled iPSC-derived secretome compositions within the physical repository derived using one or more iPSCs and/or one or more iPSC-derived cells identified as compatible with the particular subject or group of subjects.

In certain embodiments, step (c) comprises retrieving, the one or more identified iPSC-derived secretome compositions corresponding to the one or more iPS cells and/or cell lines identified as compatible with the particular subject or particular group of subjects.

In certain embodiments, step (d) comprises producing a lyophilized iPSC-derived secretome composition.

In certain embodiments, the iPSC-derived secretome composition is administered as treatment to the particular subject or particular group of subjects. In certain embodiments, the treatment is a spray. In certain embodiments, the treatment is a cream and/or lotion.

In certain embodiments, the iPSC-derived secretome composition comprises one or more proteins listed in Table 1, and/or Table 2, and/or Table 3, and/or Table 4.

In certain embodiments, the iPSC-derived secretome composition is for internal use. In certain embodiments, the iPSC derived secretome composition is an injection. In certain embodiments, the iPSC-derived secretome composition is lyophilized.

In certain embodiments, the method comprises engineering the compatible cells to upregulate and/or downregulate production of one or more desired proteins in the iPSC-derived secretome composition. In certain embodiments, the compatible cells are engineered using CRISPR/Cas9 technology. In certain embodiments, the method comprises removing and/or replacing and/or editing one or more genes of the compatible cells so as to increase the likelihood of the upregulation and/or downregulation of one or more desired proteins in the iPSC-derived secretome composition.

In certain embodiments, the iPSC-derived secretome composition comprises exosomes. In certain embodiments, the iPSC-derived secretome composition comprises microvesicles. In certain embodiments, the exosomes comprise proteins, and/or siRNAs, and/or miRNAs. In certain embodiments, the microvesicles comprise proteins, and/or siRNAs, and/or miRNAs.

In certain embodiments, the iPSC-derived secretome composition comprises one or more compatible cell types.

In another aspect, the invention is directed to a method of treating a condition in a subject, the method comprising: identifying, as compatible with the subject, an iPSC-derived secretome composition; and administering the iPSC-derived secretome composition to the subject.

In certain embodiments, the iPSC-derived secretome composition comprises one or more proteins listed in Table 1, and/or Table 2, and/or Table 3, and/or Table 4.

In certain embodiments, the step of identifying the compatible iPSC-derived secretome composition comprises the steps of: determining HLA loci, and/or ABO blood type, and/or RHD blood group associated with one or more iPSCs and/or one or more iPSC-derived cells from which the iPSC-derived secretome composition is derived; and matching, by a processor of a computing device, the determined HLA loci, and/or ABO blood type, and/or RHD blood group of the iPSC-derived secretome composition with the HLA loci, and/or ABO blood type, and/or RHD blood group of the subject, wherein a match is an exact match or a partial match.

Elements of embodiments involving one aspect of the invention (e.g., methods) can be applied in embodiments involving other aspects of the invention (e.g., systems), and vice versa.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

“Administration”: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. For example, in some embodiments, administration may be systemic or local. In some embodiments, administration may be enteral or parenteral. In some embodiments, administration may be by injection (e.g., intramuscular, intravenous, or subcutaneous injection). In some embodiments, injection may involve bolus injection, drip, perfusion, or infusion. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

“Animal”: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

“Bank”: As used herein, the term “bank” refers to a system, apparatus, or location where genetic material and/or biological sample is stored. Genetic material may be derived (e.g., extracted) from a biological sample provided by an individual to the organization that owns and/or operates the bank. In certain embodiments, biological samples are stored in a bank separate from a bank that stores genetic material extracted therefrom.

“Sample” or “Biological Sample”: As used herein, the term “sample” or “biological sample”, as used herein, refers to a biological sample obtained or derived from a source of interest, as described herein. In certain embodiments, a source of interest comprises an organism, such as a microbe, a plant, an animal, or a human. In certain embodiments, a biological sample is or comprises biological tissue or fluid. In certain embodiments, a biological sample may be or comprise bone marrow; blood; blood cells; ascites; tissue or fine needle biopsy samples; cell-containing body fluids; free floating nucleic acids (e.g., cell free DNA); sputum; saliva; urine; cerebrospinal fluid, peritoneal fluid; pleural fluid; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In certain embodiments, a biological sample is or comprises cells obtained from an individual. In certain embodiments, obtained cells are or include cells from an individual from whom the sample is obtained. In certain embodiments, a sample is a “primary sample” obtained directly from a source of interest by any appropriate means. For example, in certain embodiments, a primary biological sample is obtained by methods selected from the group consisting of a swab, biopsy (e.g., fine needle aspiration or tissue biopsy), surgery, collection of body fluid (e.g., blood, lymph, feces etc.), etc. In certain embodiments, as will be clear from context, the term “sample” refers to a preparation that is obtained by processing (e.g., by removing one or more components of and/or by adding one or more agents to) a primary sample. For example, filtering using a semi-permeable membrane. Such a processed “sample” may comprise, for example nucleic acids or proteins extracted from a sample or obtained by subjecting a primary sample to techniques such as amplification or reverse transcription of mRNA, isolation and/or purification of certain components, etc.

“Cancer”: As used herein, the terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

“Carrier”: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

“Cells” or “Cells lines”: As used herein, the term “cells” or “cells lines” refers to cells derived from human and/or non-human samples. In certain embodiments, cells can include in vitro cultured cells like iPSC-derived cells. In certain embodiments, cells can include cell lines. For example, cells can include iPSCs, and/or hematopoietic stem cells (HSCs), and/or blood progenitor cells, and/or mesenchymal stem cells (MSCs), and/or Retinal Pigment Epithelium (RPEs), and/or chondrocytes, and/or embryoid bodies, and/or any other iPSC-derived cells, and/or iPSC lines, and/or HSC lines, and/or blood progenitor cell lines, and/or MSCs lines, and/or RPE lines, and/or chondrocyte lines, and/or embryoid bodies of an iPSC line, and/or any other iPSC-derived cell lines. The cells and/or cell lines may or may not be immortalized.

“Composition”: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

“Engineered”: Those of ordinary skill in the art, reading the present disclosure, will appreciate that the term “engineered”, as used herein, refers to an aspect of having been manipulated and altered by the hand of man. In particular, the term “engineered cell” refers to a cell that has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation. In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell.

“Genotype”: As used herein, the term “genotype” refers to the diploid combination of alleles at a given genetic locus, or set of related loci, in a given cell or organism. A homozygous subject carries two copies of the same allele and a heterozygous subject carries two distinct alleles. In the simplest case of a locus with two alleles “A” and “a”, three genotypes can be formed: A/A, A/a, and a/a.

“Genotyping data”: As used herein, the term “genotyping data” refers to data obtained from measurements of a genotype. In certain embodiments, genotyping data describes an individual's phenotype. Genotyping data may be measurements of particular genes (e.g., portions of an individual's genetic sequence, e.g., DNA sequence), SNPs, or variants of SNPs. In certain embodiments, genotyping data is obtained from a multi-gene panel. In certain embodiments, genotyping data is generated in response to a purchase or request by an individual. In certain embodiments, genotyping data comprises data for a portion of a genotype (e.g., of an individual). In certain embodiments, genotyping data comprises all available measurements of a genotype (e.g., of an individual).

“Human”: In some embodiments, a human is an embryo, a fetus, an infant, a child, a teenager, an adult, or a senior citizen.

“iPSC-derived”: As used herein, the term “iPSC-derived” refers to a composition, or cell, or molecule, or element of a cell which is derived from an induced pluripotent stem cell (iPSC) and/or cell line. In certain embodiments, the composition, or cell, or molecule, or element of a cell may be derived directly or indirectly from the iPS cell and/or cell line.

“Partially un/differentiated”: As used herein, the term “partially un/differentiated” describes a biological cell that, like a state of stem cell, has a tendency to differentiate into a specific type of cell, but is already more specific than a stem cell and is pushed to differentiate into its “target” cell. For example, a difference between stem cells and progenitor cells is that stem cells can replicate indefinitely, whereas progenitor cells can divide only a limited number of times. An example of a partially undifferentiated cell is a progenitor cell.

“Reserve”: As used herein, the term “reserve” refers to an amount of biological material (e.g., cells and/or cell lines) stored in a bank.

“Subject” or “Individual”: As used herein, the term “subject” or “individual” refers to a human or other animal, or plant. In certain embodiments, subjects are humans and mammals (e.g., mice, rats, pigs, cats, dogs, horses, and primates). In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals are, for example, rodents (e.g., mice, rats, hamsters), rabbits, primates, or swine such as inbred pigs and the like.

“Secretome composition”: As used herein, the term “secretome composition” refers to a composition comprising one or more substances which are secreted from a cell. In certain embodiments, a secretome composition may include one or more cytokines, one or more exosomes, and/or one or more microvesicles. A secretome composition may be purified or unpurified. A secretome composition may further comprise one or more substances that are not secreted from a cell (e.g., culture media, additives, nutrients, etc.).

“Treatment”: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder, and/or condition, and/or of a subject who exhibits only early signs of the disease, disorder, and/or condition. Alternatively or additionally, such treatment may be of a subject who exhibits one or more established signs of the relevant disease, disorder, and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

“Variant”: As used herein, the term “variant” refers to a specific variation of a specific SNP occurring in the genome of an organism. In certain embodiments, a variant is a specific combination of a first allele of a first copy of an individual's genetic material (e.g., corresponding to an individual's paternal DNA) and a second allele of a second copy of an individual's genetic material (e.g., corresponding to an individual's maternal DNA), as occurs in diploid organisms (e.g., humans).

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader and are not intended to be limiting.

### BRIEF DESCRIPTION OF THE DRAWING

The Drawings, which are comprised of at least the following Figures, is for illustration purposes only, not for limitation.

FIG. 1 is a block diagram of an example network environment for use in the methods and systems described herein, according to an illustrative embodiment.

FIG. 2 is a block diagram of an example computing device and an example mobile computing device, for use in illustrative embodiments of the invention.

FIG. 3 is a block diagram showing a method of manufacturing an induced pluripotent stem cell (iPSC)-derived secretome composition tailored for treatment of a particular subject or particular group of subjects, according to an illustrative embodiment of the invention.

FIG. 4 is a block diagram showing a method of storing an iPSC-derived secretome composition tailored for treatment of a particular subject or particular group of subjects, according to an illustrative embodiment of the invention.

FIG. 5 is a block diagram showing a method of retrieving one or more produced, labeled and stored iPSC-derived secretome compositions derived using iPS cells and/or cell lines, according to an illustrative embodiment of the invention.

FIG. 6 is a block diagram showing a method of administering an iPSC-derived secretome composition tailored for treatment of a particular subject or particular group of subjects, according to an illustrative embodiment of the invention.

FIG. 7 is a block diagram showing a method of treating a condition in a subject, according to an illustrative embodiment of the invention.

FIG. 8 is a block diagram showing a method of manufacturing an induced pluripotent stem cell (iPSC)-derived secretome composition tailored for treatment of a particular subject or particular group of subjects, according to an illustrative embodiment of the invention.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

Presented herein are methods of producing “personalized” secretome compositions suitable for secretome based therapy (e.g., suitable for cytokine therapy and/or exosome therapy and/or microvesicle therapy) to be administered to a specific individual and/or specific group of individuals. The iPS cells and/or cell lines, iPSC-derived cells and/or cell lines, and any iPSC-derived secretome compositions and/or cytokine compositions and/or exosome compositions and/or microvesicle compositions derived therefrom, are identified as compatible with a specific individual or specific group of individuals using an identification of a cell type indicative of compatibility such as an HLA match and/or ABO blood match and/or RHD blood group match. The compatible iPS cells or cell lines (and/or cells/cell lines derived therefrom) are then retrieved from a managed HLA-indexed (and/or otherwise indexed) repository or are derived from a biological sample of a suitable donor. The retrieved compatible cells are then used to derive the “personalized” iPSC-derived secretome composition and/or cytokine composition and/or exosome composition and/or microvesicle composition, wherein the “personalized” iPSC-derived secretome composition and/or cytokine composition and/or exosome composition and/or microvesicle composition comprises the complete secretome or a subset of the secretome with the one or more desired cytokines suitable for cytokine therapy, and/or exosomes for exosome therapy, and/or microvesicles for microvesicle therapy of a specific individual and/or specific group of individuals.

In certain embodiments, secretome compositions derived from iPSCs, and/or hematopoietic stem cells (HSCs), and/or blood progenitor cells, and/or mesenchymal stem cells (MSCs), and/or Retinal Pigment Epithelium (RPEs), and/or chondrocytes, and/or embryoid bodies, and/or any other iPSC-derived cells and/or any combinations thereof are useful as therapies to treat various diseases, e.g., cancers and traumatic brain injury. In certain embodiments, cytokines, a subset of the secretome, are isolated and used in the treatment of disease or as other therapy. Cytokine therapy generally involves manipulating the immune response of the patient so as to promote immune cell generation for organ or disease treatment. iPSCs can be used in cytokine therapy to produce the desired cytokines.

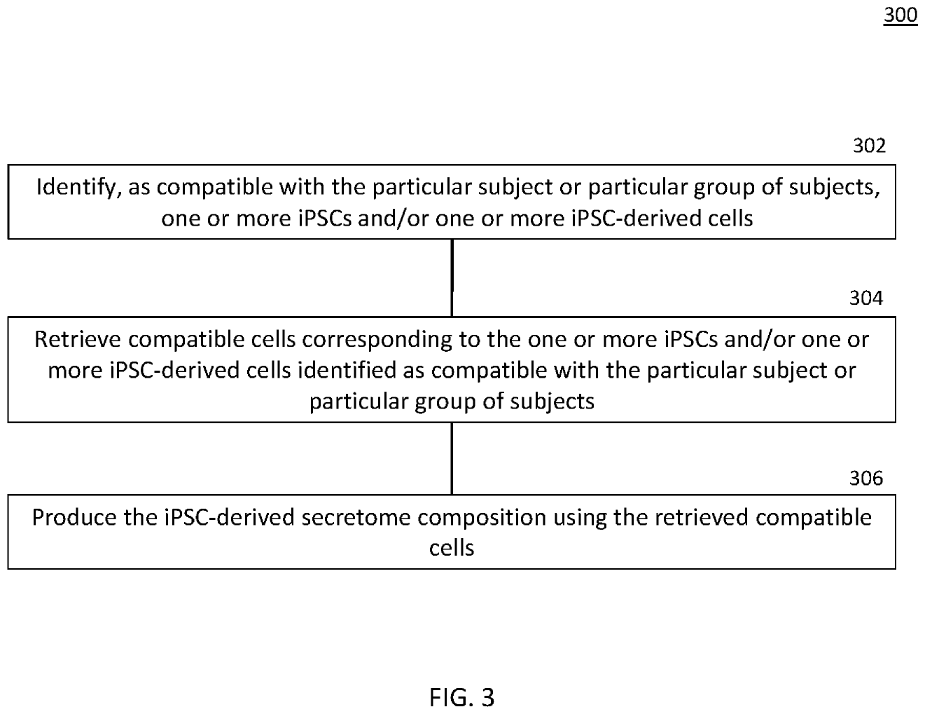

FIG. 3 is a block diagram showing a method 300 of manufacturing an iPSC-derived secretome composition, according to an illustrative embodiment of the invention. In step 302 the induced pluripotent stem (iPS) cells and/or iPSC-derived cells are identified as compatible with the particular subject or particular group of subjects. In certain embodiments, the iPS and/or iPSC-derived cells may belong to one or more cell types (e.g., HLA types), each of which is compatible with the particular subject or group of subjects. In certain embodiments one or more iPS cell lines and/or one or more iPSC-derived cell lines may also be identified, said cell lines being of one or more types (e.g., HLA types) each of which is compatible with the particular subject or group of subjects. In certain embodiments, the compatible cells and/or cell lines may be derived from the subject (e.g., autologous). In certain embodiments, the compatible cells and/or cell lines may be from an individual other than the subject (e.g., allogeneic). In step 304, the compatible cells corresponding to the one or more cells and/or cell lines identified as compatible with the particular subject or particular group of subjects are retrieved. The iPSC-derived secretome composition is then produced 306 using the retrieved compatible cells. In certain embodiments, the iPSC-derived secretome composition comprises compatible cells and one or more desired compatible-cell-secreted species (e.g., molecules and/or biological elements), (e.g., collagen, proteoglycans etc.) suitable for treatment of the subject.

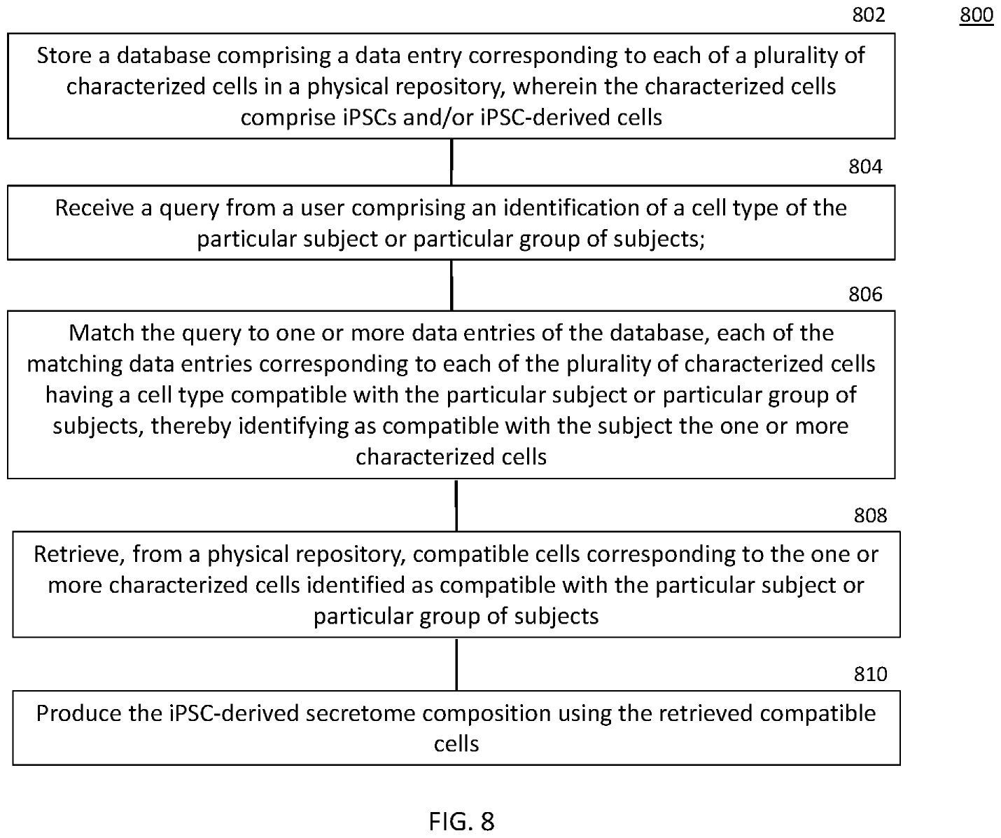

FIG. 8 is a block diagram showing a method 800 of manufacturing an induced pluripotent stem cell (iPSC)-derived secretome composition tailored for treatment of a particular subject or particular group of subjects. In step 802, a processor of a computing device, a database comprising a data entry corresponding to each of a plurality of characterized cells in a physical repository are stored. In certain embodiments, the characterized cells comprise iPSCs and/or iPSC-derived cells (e.g., HSCs, MSCs, RPEs, chondrocytes, neurons, embryoid bodies and the like). In step 804, a query from a user comprising an identification of a cell type (e.g., HLA type, and/or ABO blood group, and/or RHD blood type) of the particular subject or particular group of subjects is received by the processor. In certain embodiments, the query may additionally comprise of ABO blood group, and/or RHD blood type. Then the query is matched (806) by the processor to one or more data entries of the database. Each of the matching data entries corresponds to each of the plurality of characterized cells (e.g., iPSCs, and/or iPSC-derived cells (e.g., HSCs, MSCs, RPEs, blood progenitors, chondrocytes, neurons, and embryoid bodies)), and/or iPSC lines, and/or iPSC-derived cell lines) having a cell type compatible with the particular subject or particular group of subjects. Each of the plurality of characterized cells corresponding to the matched data entries are identified as compatible with the particular subject or particular group of subjects. In step 808, the compatible cells corresponding to the one or more characterized cells identified as compatible with the particular subject or particular group of subjects are retrieved from a physical repository. The iPSC-derived secretome composition is then produced (810) using the retrieved compatible cells. In certain embodiments, the iPSC-derived secretome composition comprises compatible cells and one or more desired compatible-cell-secreted species (e.g., molecules and/or biological elements), (e.g., collagen, proteoglycans etc.) suitable for treatment of the subject.

The techniques described herein allow for the tuning of secretome compositions to a specific individual or a specific group of individuals, thus enabling improved methods of secretome based therapy, e.g. due to an enhanced compatibility of the specific individual or group of individuals with the cells from which the desired secretome composition is derived. Also, allogeneic iPS cells and/or cell lines that are compatible with a large portion of a specific population, e.g. super donors, can be prepared and stored in advance for large groups of individuals. These super donor-derived secretome compositions can then be made immediately available to people who need them, thus reducing production times of the iPSC-derived secretome compositions.

iPSCs, or cells differentiated from iPSCs, can be made to produce a desired secretome, e.g., which comprises desired cytokines. For example, secretome can produced from iPSCs of a super donor cell line. Secretome can also be produced from MSCs, HSCs, RPEs, chondrocytes, or other cell types derived from iPSCs. In certain embodiments, allogeneic iPSCs (and/or cells derived therefrom) and/or allogeneic iPSC-derived secretome compositions can be prepared and stored for large groups of individuals. Allogeneic iPSCs (and/or cells derived therefrom) and/or iPSC-derived secretome compositions can be made in advance so that they are ready when people need them. For example, the iPSCs, and/or iPSC-derived cells and/or iPSC-derived secretome compositions can be lyophilized and stored for later use.

In certain embodiments, iPSCs (and/or cells derived therefrom) and/or iPSC-derived secretome compositions can be lyophilized to manufacture a more concentrated solution or composition. In certain embodiments, iPSCs, or cells differentiated from iPSCs, can be engineered using various technologies (e.g., CRISPR/Cas9) to upregulate production of one or more desired proteins in the secretome. For example, in certain embodiments, an iPS cell (and/or cells derived therefrom) may be edited via CRISPR (e.g., CRISPR-Cas9 genome editing and/or gene transfer) to remove, replace, and/or edit one or more genes to result in (or to increase the likelihood of) the upregulation of one or more desired proteins in the secretome of the iPSCs and/or cells derived therefrom.

In certain embodiments, the invention is directed to a managed repository of secretome compositions, cytokine compositions, hematopoietic stem cell (HSC) lines and/or blood progenitor cell lines, RPE lines, MSC lines, chondrocyte lines and/or other cell lines derived from induced pluripotent stem cells (iPSCs) (e.g., embryoid bodies or other tissues formed from iPSCs). In certain embodiments, the secretome compositions, cytokine compositions, HSC lines, blood progenitor cell lines, embryoid bodies, RPE lines, MSC lines, chondrocyte lines, iPSC lines and/or iPSC-derived cell lines has corresponding data comprising a set of characterized HLA loci, said corresponding data being stored in a searchable database for retrieval of one or more matching physical cell lines and/or cytokine compositions upon query. The repository may comprise a bank of cells (e.g., iPSCs, HSCs, blood progenitor cells, embryoid bodies, RPEs, MSCs, chondrocytes, other iPSC-derived cells), and/or compositions produced from cells, for each of a set of HLA types. This allows identification and provision of existing compatible iPSC-derived secretome compositions, iPSC-derived cytokine compositions, iPSC-derived exosome compositions, iPSC-derived microvesicle compositions, iPSCs, embryoid bodies, RPEs, MSCs, chondrocytes, HSCs, blood progenitor cells, and/or other iPSC-derived cells for a particular subject or group of subjects. The iPSC-derived secretome, cytokine, exosome, and/or microvesicle compositions—and allogeneic cell lines (e.g., iPSC lines, MSC lines, RPE lines, chondrocyte lines, HSC lines, blood progenitor cell lines, other iPSC-derived cell lines) suitable for deriving secretomes, cytokines, exosomes, and microvesicles—can be used to formulate compositions for administration topically or internally (e.g., injection, parenteral, oral, rectal, vaginal etc.) to regenerate, treat, and/or cosmetically enhance skin and/or other organs in patients with damaged, diseased, or otherwise abnormal organs. For example, iPSCs, iPSC-derived cells (e.g., HSCs, blood progenitor cells, embryoid bodies, RPEs, MSCs, chondrocytes, other iPSC-derived cells), iPSC-derived composition (e.g., secretome composition, cytokine composition, microvesicle composition, and/or exosome composition), and/or combinations therefrom can be administered via an injection (e.g., subcutaneous, intramuscular, etc.) to tissue that have low vasculature (e.g., around joints) to aid in repair of the tissue. In certain embodiments, the administered solution of cells, compositions and/or combinations therefrom may include additives (e.g., nutrients to keep cells alive/active before, during, and/or after administration, carriers, fillers etc.).

### Human Leukocyte Antigen

The characterized iPS cells and/or cell lines and/or compositions derived therefrom are stored in the repository that is indexed using the Human Leukocyte Antigen (HLA). In certain embodiments, the iPS cells and/or cell lines and/or compositions derived therefrom are characterized and indexed as super donor cell lines via HLA mapping (e.g., HLA typing and/or matching). In certain embodiments, multiple HLA loci may be characterized and indexed for each of the various iPS cells and/or cell lines and/or cells derived therefrom and/or compositions derived therefrom.

The HLAs in humans are major histocompatibility complex (MHC) proteins that function to regulate the immune system. HLA genes are highly polymorphic and may be broadly divided into Class I and Class II. For example, Class I in humans may be found on all nucleated cells and platelets. On the other hand, HLA Class II (constitutive expression), for example, may be restricted to specialized cells of the immune system (e.g., macrophages, B cells, etc.).

HLA Class I, for example, may include HLA-A, B, and C genes. In certain embodiments, HLA Class I may be co-dominantly expressed on the cell surface and may present peptides derived from internal cellular proteins to the T cell receptor of CD8 T cells. For example, these proteins may be involved in the immune response against intracellular parasites, viruses, and cancer.

In certain embodiments, HLA Class I may have a heterodimeric protein structure, with a polymorphic alpha chain and a common beta-2 microglobulin. In certain embodiments, the alpha chain may be composed of 3 extracellular domains: α1, α2, and α3.

HLA Class II, for example, may include DR, DQ and DP genes. In certain embodiments, HLA Class II may be co-dominantly expressed. In certain embodiments, HLA Class II may have a heterodimeric protein structure, with a polymorphic beta chain and a much less polymorphic alpha chain. In certain embodiments, both chains may be composed of two (2) extracellular domains (α1, α2, and β1, β2). For example, the α1 and β1 domains may create a peptide binding groove which presents processed peptides, from extracellular protein, to CD4+ T cells. In certain embodiments, HLA Class II may be involved in the immune response against extracellular infectious agents and non-self HLA molecules.

In certain embodiments, each HLA allele may be identified by letters indicating “locus” (e.g., A, B, C, DR, DQ, and DP) and individual specificity may be defined by a number following the locus (e.g., A1, B27, DR8, etc.). Specificities can be defined using antisera (antibodies). In certain embodiments, HLA specificities may also be determined using genetic analysis by identifying the presence/absence of the gene encoding the HLA protein. For example, Class II molecular specificities may be identified at the level of the gene encoding a particular chain (α or β).

### HLA Typing

The stem cells and/or stem cell lines (e.g., iPSCs) and/or cells derived therefrom and/or compositions derived therefrom stored in the physical repository may be characterized and indexed using various characteristics of the samples (e.g., cells). In certain embodiments, the stems cells and/or cell lines and/or cells derived therefrom and/or compositions derived therefrom may be characterized and indexed using HLA type. In certain embodiments, the stems cells and/or cell lines and/or cells derived therefrom and/or compositions derived therefrom may be characterized and indexed using ABO blood group. In certain embodiments, the stems cells and/or cell lines and/or cells derived therefrom and/or compositions derived therefrom may be characterized and indexed using RHD blood type. For example, the stems cells and/or cell lines and/or cells derived therefrom and/or compositions derived therefrom may be characterized and indexed in the physical repository using HLA type, and/or ABO blood group, and/or RHD type.

HLA typing or HLA matching is used to determine the HLA type of an individual. The HLA type of an individual comprises a pair of co-expressed haplotypes, each corresponding to a set of HLA genes (e.g., an HLA-A, an HLA-B, and an HLA-DR gene). In certain embodiments, genetic recombination and environmental factors result in linkage disequilibrium with respect to inheritance of HLA gene combinations. For example, certain combinations of HLA alleles (e.g., combinations of HLA-A, -B, and -DR genes) are favored, whereas other combinations do not exist.

HLA typing may be performed at a protein level but may also be performed at the DNA level, for example by amplifying the DNA via polymerase chain reaction (PCR), or other DNA identification and amplification technologies. For example, HLA typing may be performed using sequence specific oligonucleotides (SSO). In certain embodiments, SSO-based HLA typing may use generic primers to amplify large amounts of HLA alleles, for example, HLA-A, via PCR or other DNA amplification technologies. The dsDNA is separated into single strands and allowed to interact with the single strand specific oligonucleotide probes. In certain embodiments, such probes may be bound to a solid matrix. For example, the pattern of the bound probes may be used to determine the HLA type of the specimen. In certain embodiments, HLA typing may be performed using sequence specific primers (SSP). For example, in SSP-based HLA typing amplifies DNA that matches the primers. Antibodies may also been used for HLA typing, but may have the disadvantage of cross-reacting with multiple HLA epitopes (e.g. HLA-A2, A9 and A28).

### Applications of HLA Typing

The HLA type of a sample (e.g., cells, organs, and/or tissue) may be used in determining compatibility between organ donors and recipients. Samples which match the HLA type of a recipient (e.g., patient) are more likely to not illicit an immune response (e.g., rejection) after the sample is transplanted to the recipient. In certain embodiments, matching is performed on the basis of 3 or more loci on the HLA gene to prevent a strong immune response in the recipient post transplantation. In certain embodiments, at least 3 HLA loci are required to match between the donor and the recipient to prevent a strong immune response in the recipient post transplantation. In certain embodiments, at least 3, or at least 4, or at least 5, at least 6, or at least 7, or at least 8, or at least 9 major sites (e.g., loci) are required to match between the donor and the recipient to prevent a strong immune response in the recipient post transplantation.

Many registry donors have been tested by serological (e.g., HLA mapping using antigens) methods, though often without documentation regarding which antigens were tested. While the majority of hematopoietic progenitor cell transplant candidates have been tested by molecular (DNA-based) methodologies, the nomenclature of antigens (serology) and alleles (DNA) is in some cases not concordant. Thus, the characterized and indexed (e.g., HLA indexed (e.g., using standard nomenclature)) iPS cells and/or cell lines and/or cells derived therefrom and/or compositions derived therefrom, described herein, may be used to efficiently and accurately searched using the corresponding database to quickly find matching HLA samples for implantation. For example, the HLA indexed and matched iPS cells and/or cell lines and/or cells derived therefrom and/or compositions derived therefrom may be used in treatment of various diseases. In certain embodiments, these cells and/or cell lines may be used in the treatment cancer (e.g., leukemia, lymphoma, bone cancer, and the like). In certain embodiments, these cells and/or cell lines may be used in Hematopoietic stem cell transplantation.

The HLA-indexed repository may also be used for various purposes. For example, other clinical applications of HLA typing may include disease risk assessment, pharmacogenomics, immunotherapy, infectious disease vaccines, and tumor vaccines. In certain embodiments, the cells and/or cell lines stored and indexed in the repository may be used in cosmetic surgery, for example cartilage grafts. Long-term transplant and graft survival is correlated to the degree of HLA antigen mismatch for both solid organ and bone marrow transplant.

HLA matched cells and/or cell lines may also be used in the treatment of various diseases. Certain diseases may have a strong association with certain specific HLA types. For example, HLA associations with diseases include ankylosing spondylitis and acute anterior uveitis (HLA-B27); birdshot retinopathy (HLA-A29); Behçet's Disease (HLA-B51); psoriasis (HLA-Cw6); celiac disease (HLA-DQ2,8); narcolepsy (HLA-DR15, DQ6); diabetes (HLA-DR3,4-DQ2,8); and rheumatoid arthritis (HLA-DR4). In certain embodiments, the data entries in the HLA database corresponding to specific samples (e.g., cells and/or cell lines in the physical repository) may incorporate information regarding their specific HLA types to recognize their strong associations with certain diseases.

HLA type may also be associated with allergy or hypersensitivity to a medication. For example, severe allergic or hypersensitivity reaction to drugs in Stevens-Johnson Syndrome (SJS) and toxic epidermal necrolysis (TEN) may be associated with HLA type. The physical repository of cells and/or cells lines and corresponding database may be used to identify allergies and sensitivities in the patients (e.g., sometimes unknown to the patient). In certain embodiments, HLA typing allows risk stratification of the patients. In certain embodiments, drugs that are associated with hypersensitivity reactions (e.g., antiepileptic agents, allopurinol, nevirapine, anti-inflammatories in oxicam family, and sulfonamides) may be studied using the cells and/or cell lines and/or cells derived therefrom stored in the repository. Further, these studies can be performed in vitro and/or ex vivo prior to implantation.

HLA typing may be used for vaccine development. The HLA-indexed cells and/or cell lines and/or cells derived therefrom and/or compositions derived therefrom described herein may be used to develop such vaccines. In certain embodiments, vaccines producing cellular immunity require peptide HLA binding. For example, vaccine trials use peptides binding to common HLA alleles. After proof-of-principal, trials may include peptides binding to other HLA alleles. In certain embodiments, cells with the common HLA allele, and cells with other HLA alleles may be selected from the back of stem cells and/or cell lines stored in the repository.

HLA typing can also be informative for compatibility of individuals. For example, studies have found that husbands and wives have fewer HLA matches than expected. The HLA genes (HLA-A, HLA-B, and HLA-DRB1) regulate the immune system, and thus determine the microbes that the immune system attacks. As a non-limiting example, the HLA genes therefore regulate a subject's smell by governing the non-human microbes associated with that subject and therefore can affect the attraction between subjects based on smell, among other things. Given the association between HLA type and long-term compatibility, it may be possible to predict the likelihood of companionship between two individuals. In some embodiments, the present disclosure teaches a method of querying and retrieving data entries of a database matching queried HLA loci for compatibility or companionship for a given subject with other individuals.

### HLA-Indexed Bank

The bank of iPS cells and iPSC-derived compositions (e.g., IPS cells and/or cell lines and/or cells derived from iPSCs (e.g., HSCs and/or blood progenitors) and/or secretome compositions derived from iPSCs and/or CAR-T compositions derived from iPSCs) is a comprehensive indexed repository in that it contains a variety of HLA types covering a significant proportion (e.g., at least 85%, at least 90%, or at least 95%) of a given population, indexed by HLA type and/or ABO group and/or RHD type. In certain embodiments, the HSC lines and/or blood progenitors in the bank (and/or the iPS cell lines and/or embryoid bodies from which the HSCs and/or blood progenitors are derived), may be characterized as super donor cell lines (e.g., via HLA mapping). Thus, it is possible to obviate the need for bone marrow registries and/or other donor registries, since suitable cells for transplantation may be quickly identified and made available to patients over a wide swath of a given population upon demand, without the difficult, time consuming process of identifying a matching blood marrow donor. Identification of a suitable cell line may include matching the patient's ABO blood type and/or RHD blood group to that of the HSC, blood progenitor cell, embryoid body, and/or iPSC line, in addition to HLA type.

The bank may provide access to reserves of immortalized iPSCs from which iPSC secretome compositions can be derived—iPSCs and secretome compositions derived from iPSCs may be prepared in advance for commonly-used/matched HLA types (e.g., HLA superdonors matching higher percentages of the population) so that cells and/or compositions are available immediately upon need. HSCs may also be produced for a particular patient upon identification of a matching iPSC line. Furthermore, in certain embodiments, reserves of embryoid bodies, corresponding to characterized iPSC lines, are stored in the bank. In certain embodiments, HLA superdonor lines are physically represented in the bank by embryoid bodies (characterized as HLA superdonor lines). These embryoid bodies may be used to make HSCs and/or blood progenitors.

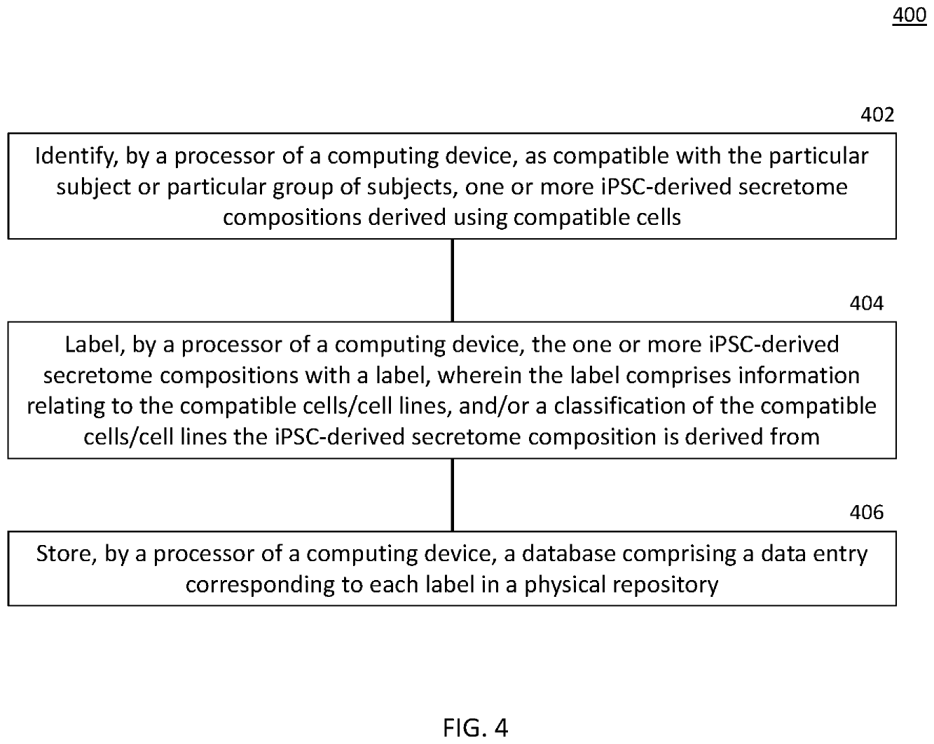

FIG. 4 is a block diagram showing a method 400 of storing an iPSC-derived secretome composition, according to an illustrative embodiment of the invention. In step 402, one or more iPSC-derived secretome compositions derived using compatible cells are identified, by a processor of a computing device, as compatible with the particular subject or particular group of subjects. In certain embodiments, the compatible cells correspond to one or more iPS (or iPSC-derived (e.g., MSC, HSC, RPE and the like)) cells and/or cell lines, said cells and/or cell lines being of one or more types (e.g., HLA type) each of which is identified as compatible with the particular subject or group of subjects. In step 404, the one or more iPSC-derived secretome compositions are labeled, by a processor of a computing device, with a label. In certain embodiments, the label may be a digital label, wherein the label comprises information relating to the iPS and/or iPSC-derived cell and/or cell line, and/or a classification of the iPS cell and/or cell line (e.g., HLA loci, and/or ABO blood type, and/or RHD blood group) the iPSC-derived secretome composition is derived from. The one or more labeled iPSC-derived secretome compositions are then stored (406), by a processor of a computing device, in a database comprising multiple data entries. One or more data entries in the database corresponds to each labeled iPSC-derived secretome compositions (e.g., or other labeled entities like cells, cell lines, other compositions and the like) stored in a physical repository.

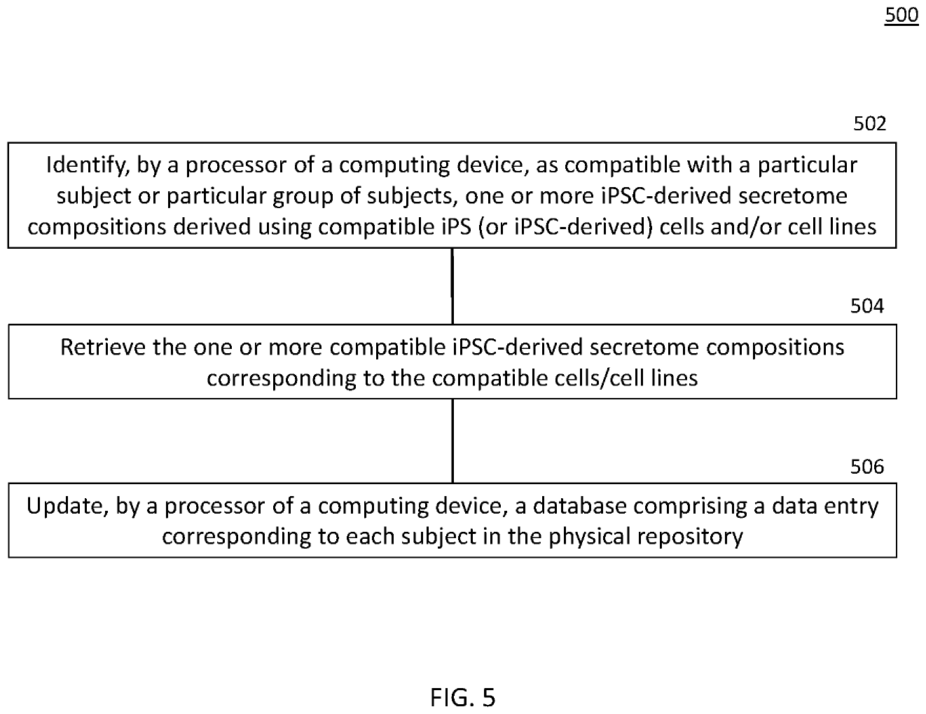

FIG. 5 is a block diagram showing a method 500 of retrieving one or more produced, labeled and stored iPSC-derived secretome compositions, according to an illustrative embodiment of the invention. In step 502, one or more iPSC-derived secretome compositions are identified, by a processor of a computing device, as compatible with a particular subject or particular group of subjects. The one or more iPSC-derived secretome compositions are derived using one or both of (i) and (ii) as follows: (i) one or iPS cells and/or iPSC-derived cells, said cells being of one or more types (e.g. HLA type) each of which is identified as compatible with the particular subject or group of subjects, and (ii) one or more iPS cell lines and/or one or more iPSC-derived cell lines, said cell lines being of one or more types each of which is identified as compatible with the particular subject or group of subjects. In step 504, the one or more compatible iPSC-derived secretome compositions corresponding to the one or more iPS and/or iPSC-derived cells and/or cell lines identified as compatible with the particular subject or particular group of subjects are retrieved (e.g., from the physical repository in which the one or more iPSC-derived secretome compositions are stored). The database data entry of each subject of the group of subjects is then updated (506), by a processor of a computing device. The update to the data entry corresponding to each subject may include identification information (e.g., label information) regarding the one or more iPSC-derived secretome compositions in the physical repository that each subject is compatible with.

Induced human pluripotent stem cells (iPSCs) can be generated from biological samples, such as blood samples. Depending on the conditions, in vitro iPSCs can retain their pluripotency or they can be directed to differentiate into a wide range of specialized cell types and tissues. Such cell types and tissues can be used for applications including replacement of diseased or damaged tissues in patients with conditions such as trauma, diabetes, degenerative neurological disorders, cardiovascular disease, and metabolic deficiencies.

As discussed in Taylor et al., Cell Stem Cell 11, Aug. 3, 2012, pp. 147-152, the contents of which are incorporated herein by reference, HLA-mismatched iPSCs can cause immunological rejection and therefore limit therapeutic potential. iPSCs derived directly from patients (autologous iPSCs) can result in matched HLA type and reduce risk of transplant rejection. However, generation of autologous iPSCs for individual patients is costly and time-consuming. Alternatively, allogeneic iPSC cell lines with HLA types that do not trigger strong reactions can be prepared and used for large groups of individuals.

The term “super donor” is a term used to describe HLA types that do not trigger strong rejection reactions. Such allogeneic (derived from donors other than the patient) iPSC lines can be made in advance and can be ready for use when needed. Fewer allogeneic lines are needed to serve a population. iPSCs can be obtained from healthy volunteer donors of blood group O that are selected to maximize the opportunity for HLA matching. Clinical grade iPSC lines can be expanded and differentiated for use in a large number of subjects. Nakajima et al., Stem Cells 25, 2007, pp. 983-985, the contents of which are incorporated by reference herein, discusses HLA matching estimations in a hypothetical bank of human embryonic stem cell lines in the Japanese population, and calculated that a large proportion of patients were able to find at least one HLA matched donor at three loci of HLA-A, HLA-B, and HLA-DR for transplantation therapy.

Because the iPSC lines, MSC lines, RPE lines, chondrocyte lines, HSC lines, blood progenitor cell lines, and/or other iPSC-derived cell lines are characterized by HLA type, an iPSC line, MSC line, RPE line, chondrocyte line, HSC line, blood progenitor cell line, other iPSC-derived cell lines and/or iPSC-derived secretome compositions can be identified as suitable for a given patient with a compatible HLA type, with low, reduced, or zero chance of a compatible cell-derived composition rejection. In certain embodiments, the bank of iPSCs, embryoid bodies, MSCs, RPEs, chondrocytes, HSCs, blood progenitor cells, other iPSC-derived cells and/or compositions derived therefrom is comprehensive in that it contains a variety of HLA types covering a significant proportion (e.g., at least 85%, at least 90%, or at least 95%) of a given population. In certain embodiments, the iPSC lines, MSC lines, RPE lines, chondrocyte lines, the HSC lines, blood progenitor cell lines, other iPSC-derived cell lines and/or secretome, cytokine, exosome, and microvesicle compositions in the bank and/or the iPS cell lines and/or embryoid bodies from which the MSCs, RPEs, chondrocytes, HSCs, blood progenitor cells, other iPSC-derived cells, and/or the secretome, cytokine, exosome and microvesicle compositions are derived, are characterized as super donor cell lines (e.g., via HLA mapping). Thus, suitable cells (e.g., iPSCs, iPSC-derived cells), cell lines (e.g., iPSC lines, iPSC-derived lines), iPSC-derived secretome compositions, iPSC-derived cytokine compositions, iPSC-derived exosome compositions, and/or iPSC-derived microvesicle compositions for treatment may be quickly identified and made available to patients over a wide swath of a given population upon demand, without the difficult, time consuming process of identifying a matching donor. Identification of a suitable cell line, iPSC-derived secretome composition, iPSC-derived cytokine composition, iPSC-derived exosome composition, and/or iPSC-derived microvesicle composition may include matching the patient's ABO blood type and/or RHD blood group to that of the HSC, blood progenitor cell, embryoid body, MSC, RPE, chondrocyte, other iPSC-derived cell, iPSC, secretome composition, cytokine composition, exosome composition, and/or microvesicle composition in addition to HLA type.

In certain embodiments, the bank may provide access to reserves of immortalized iPSCs from which MSCs, RPEs, chondrocytes, HSCs, blood progenitor cells, other iPSC-derived cells, secretome compositions, cytokine compositions, exosome compositions, and/or microvesicle compositions can be derived. MSCs, RPEs, chondrocytes, HSCs, blood progenitor cells, embryoid bodies, other iPSC-derived cells, and/or tissues expressing specific secretomes, cytokines, exosomes and/or microvesicles may be prepared in advance for commonly-used/matched HLA types (e.g., HLA superdonors matching higher percentages of the population) so that the compositions are available immediately upon need. These compositions may also be produced for a particular patient upon identification of a matching iPSC line.

Furthermore, in certain embodiments, reserves of embryoid bodies, corresponding to characterized iPSC lines, are stored in the bank. In certain embodiments, HLA superdonor lines are physically represented in the bank by embryoid bodies (characterized as HLA superdonor lines). These embryoid bodies may be used to make MSCs, RPEs, chondrocytes, HSCs, blood progenitor cells, and/or other iPSC-derived cells that are used to express the desired secretome with the desired cytokines and/or exosomes and/or microvesicles, used to formulate the secretome composition.

The characterized iPSCs and/or embryoid bodies comprising embryonic stem cells (e.g., undifferentiated pluripotent cells) can be differentiated into hematopoietic cells such as HSCs, hematopoietic progenitor cells, and mature hematopoietic cells (e.g. immune cells like macrophages, B lymphocytes, T lymphocytes and mast cells), MSCs, RPEs, chondrocytes, fibroblasts, various stromal cells, and other iPSC-derived cells, and made to produce various secretome compositions in the presence of appropriate culture media. In certain embodiments, the characterized cell types contained in the physical bank include any one or more of the following: iPSCs, embryoid bodies, HSCs, blood progenitor cells, mature hematopoietic cells, MSCs, RPEs, chondrocytes, and/or other iPSC-derived cells.

Matching HLA type may involve, for example, querying and retrieving data entries of a database matching queried HLA loci. In certain embodiments, this comprises receiving, by a processor of a computing device (e.g., a server), a data entry for an individual for which a matching iPSC line, and/or MSC line, and/or chondrocyte line, and/or RPE line, and/or HSC line, and/or blood progenitor line, and/or any other iPSC-derived cell line, and/or iPSC-derived secretome composition is desired, the data entry comprising a set of characterized HLA loci corresponding to the individual [e.g., identification (e.g., by processing and analyzing (e.g. by serology, by PCR) samples from the individual (e.g., blood samples)) of each of a set of at least 3 given loci (e.g., HLA-A, HLA-B, and HLA-DRB (e.g., HLA-DRB1)), e.g., at least 9 given loci (e.g., HLA-A, HLA-B, HLA-C, HLA-DRB1, HLA-DRB3, HLA-DRB4, HLA-DRB5, HLA-DQB1, HLA-DPB1), e.g., at least 3, 4, 5, 6, 7, 8, or 9 members selected from this group of nine loci]; and retrieving, by the processor, one or more data entries of a database representative of cells (e.g., iPS cells in the physical repository and/or embryoid bodies, MSCs, RPEs, chondrocytes, HSCs, blood progenitor cells, and/or other cells from a cell line derived from iPSCs), and/or iPSC-derived secretome compositions matching (e.g., exactly matching, partially matching, identified as compatible with (e.g., compatible HLA types), etc.) the queried HLA loci (e.g., determining the corresponding bar code or other identifier for the iPSCs, and/or iPSC-derived cells, and/or embryoid bodies corresponding to the data entry, thereby allowing retrieval of desired stem cells and/or secretomes from the repository and/or retrieval of identifying information corresponding to a desired iPSC cell line matching the queried HLA loci). iPSC-derived secretome compositions may be produced from immortalized iPSC lines at will and made available for ready access when needed—no additional harvesting of samples are required to produce additional iPSC-derived secretome compositions.

The repository/bank of cells and compositions may comprise a storage system comprising an insulated container equipped with environmental control system (for control of temperature, humidity, pressure, and the like) suitable to store cells (e.g., iPSCs, embryoid bodies, RPEs, chondrocytes, MSCs, HSCs, blood progenitor cells, mature hematopoietic cells, and/or other iPSC-derived cells), and secretome compositions (e.g., derived from iPSCs, embryoid bodies, MSCs, RPEs, chondrocytes, HSCs, blood progenitor cells, mature hematopoietic cells, and/or other iPSC-derived cells) for a period of time. The repository/bank may also include one or more processors (e.g., of a server) and/or related software to manage inventory, as well as a sample location system and/or retrieval system for identification/retrieval of cells and/or specific secretome compositions from a matched cell line. iPSCs may be produced from blood samples (or other biological substance sample, e.g., saliva, serum, tissue, cheek cells, cells collected via a buccal swab, urine, and/or hair), then labeled (physically and/or digitally), logged in an inventory database, and stored in the repository for ongoing and/or future use. MSCs, RPEs, chondrocytes, HSCs, blood progenitor cells, mature hematopoietic cells and/or other cell types may be produced from iPSCs via known methods. These iPSCs or iPSC-derived cells are made to produce desired secretomes that are formulated into compositions, and the iPSC-derived cells and/or secretome compositions may also be labeled (physically and/or digitally), logged in the inventory database, and stored in the repository for ongoing and/or future use.

The repository/bank of cells may be used in systems and methods for regeneration, treatment, and/or cosmetic enhancement of subjects in need of secretome therapy. For example, the repository/bank of cells comprise iPSCs and/or embryoid bodies corresponding to/produced from iPSC lines, wherein MSCs, RPEs, chondrocytes, HSCs, blood progenitor cells, and/or other cell types are derived from/produced from the iPSCs and/or embryoid bodies, and the MSCs, RPEs, chondrocytes, HSCs, blood progenitor cells, other iPSC-derived cells, iPSCs and/or embryoid bodies are utilized to derive specific secretomes that are formulated into compositions, and the secretome compositions are administered to subjects at risk of or having a disease, traumatic injury, and/or condition, such as any of the following: lung disease (e.g., Bronchopulmonary dysplasia (BPD), rheumatic diseases (e.g., rheumatoid arthritis (RA), osteoarthritis (OA)), cardiovascular disease (e.g. Acute myocardial infraction, ischemic heart disease), cancer (e.g., breast cancer), arthritis, traumatic brain injury, central nervous system (CNS) injury, and inflammation.

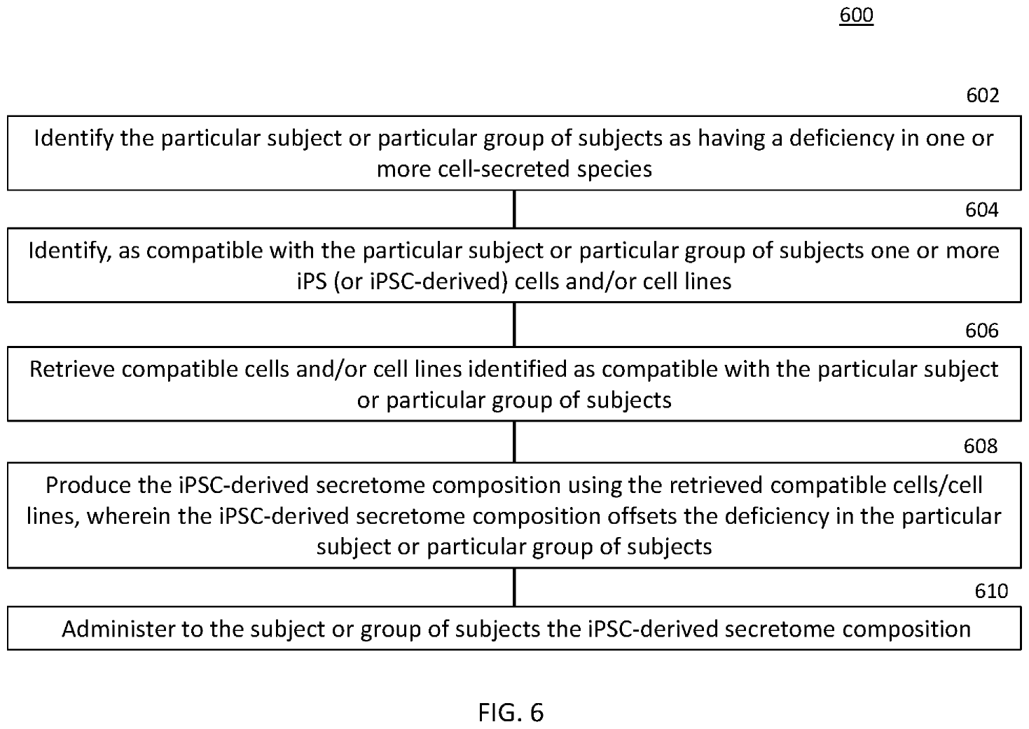

For example, FIG. 6 is a block diagram showing a method 600 of administering an iPSC-derived secretome composition tailored for treatment of a particular subject or particular group of subjects, according to an illustrative embodiment of the invention. In step 602, the particular subject or particular group of subjects as having a deficiency in one or more cell-secreted species (e.g., one or more cell-secreted molecules and/or cell-secreted biological elements) is/are identified. In second step 604, one or both of (i) and (ii) as follows: (i) one or more induced pluripotent stem (iPS) cells and/or iPSC-derived cells, said cells being of one or more types each of which is compatible with the particular subject or group of subjects, and (ii) one or more iPS cell lines and/or one or more iPSC-derived cell lines, said cell lines being of one or more types each of which is compatible with the particular subject or group of subjects, are identified as compatible with the particular subject or particular group of subjects. Following identification, in step 606, the compatible cells corresponding to the iPS and/or iPSC-derived cells and/or cell lines identified as compatible with the particular subject or particular group of subjects are retrieved (e.g., from a physical repository). The iPSC-derived secretome composition is then produced (608) using the retrieved compatible cells. The iPSC-derived secretome composition produced is engineered and/or selected such that it offsets the deficiency in the particular subject or particular group of subjects (e.g., wherein the iPSC-derived secretome composition comprises the identified one or more deficient cell-secreted species (e.g., cell-secreted molecules and/or cell-secreted biological elements identified as deficient in the subject). The iPSC-derived secretome composition is then administered (610) to the subject or group of subjects.

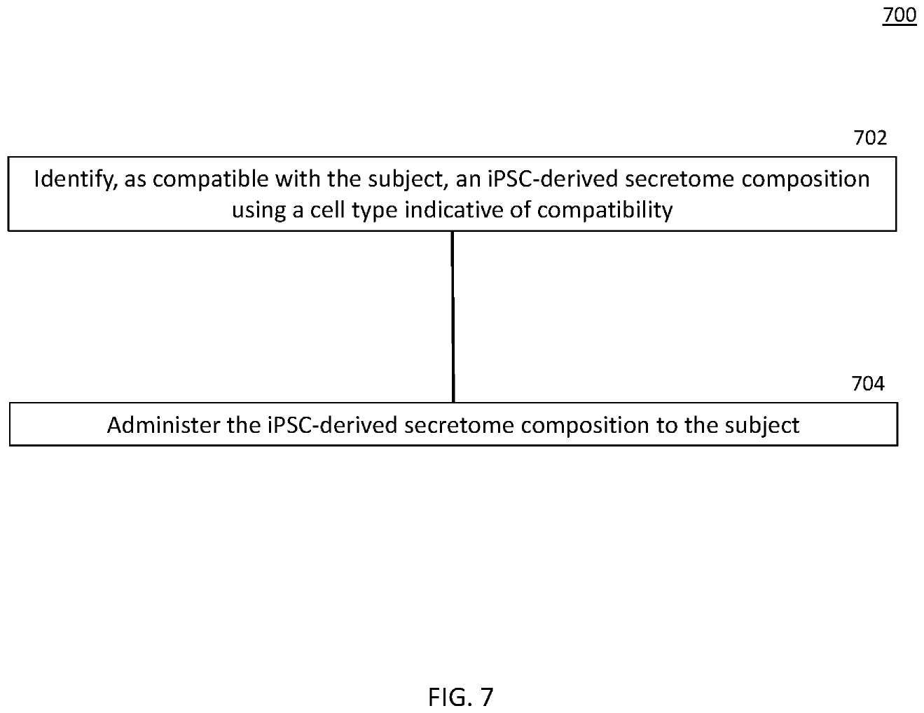

FIG. 7 is a block diagram showing a method 700 of treating a condition in a subject, according to an illustrative embodiment of the invention. In one step 702, an iPSC-derived secretome composition is identified as compatible (e.g., most compatible) with the subject using a cell type indicative of compatibility (e.g., by determining that the HLA loci, and/or ABO blood type, and/or RHD blood group associated with the cell(s) from which the iPSC-derived secretome composition is derived are identical to the HLA loci, and/or ABO blood type, and/or RHD blood group of the subject). The identified iPSC-derived secretome composition is then administered (704) to the subject.

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

Generation and Differentiation Protocols for Immortalized iPSCs

Induced pluripotent stem cell (iPSC) generation protocols are described, for example, at https://www.thermofisher.com/us/en/home/references/protocols/cell-culture/stem-cell-protocols/ipsc-protocols.html, the contents of which is hereby incorporated by reference in its entirety. Induced pluripotent stem cell (iPSC) generation and differentiation protocols are described, for example, at http://www.sigmaaldrich.com/life-science/stem-cell-biology/ipsc/ipsc-protocols.html, the contents of which is hereby incorporated by reference in its entirety. Differentiation of iPSCs can be found, for example, in “Induction of Pluripotent Stem Cells from Adult Human Fibroblasts by Defined Factors”; Takahashi K., Tanabe K., Ohnuki M., Narita M., Ichisaka T., Tomoda K., Yamanaka S.; Cell Vol. 131, 861-872, November 2007″, the contents of which is hereby incorporated by reference in its entirety.

Recently, HSCs have been successfully produced from iPSCs. See, for example, “Generation of engraftable hematopoietic stem cells from induced pluripotent stem cells by way of teratoma formation,” Mol Ther. 2013 July; 21(7); 1424-31; Epub May 14, 2013; “Hematopoietic stem cells meet induced pluripotent stem cells technology,” Haematologica, 2016 September; 101(9): 999-1001; and “In vivo generation of transplantable human hematopoietic cells from induced pluripotent stem cells,” Blood, 2013 Feb. 21; 121(8); 1255-64; Epub Dec. 4, 2012; the contents of each of which are incorporated herein by reference. Furthermore, in recent years, there have been significant advances in the production of iPSCs from cells collected from a biological sample of a subject (e.g., blood cells). For example, iPSCs can be made by inserting copies of stem cell-associated genes—e.g., Oct 3/4, Sox 2, Klf4, and c-Myc (or Oct 3/4, Sox 2, Nanog, and Lin28)—into cells collected from the biological sample using viral vectors. See, for example, K. Okita, T. Ichisaka, and S. Yamanaka, “Generation of germline-competent induced pluripotent stem cells,” Nature, vol. 448, no. 7151, pp. 313-317, 2007; K. Okita, Y. Matsumura, Y. Sato et al., “A more efficient method to generate integration-free human iPS cells,” Nature Methods, vol. 8, no. 5, pp. 409-412, 2011; the contents of each of which are incorporate herein by reference.

Storage of Immortalized iPSCs

Repositories (290) (e.g., cell repositories, e.g., nucleic acid repositories) for storing biological sample material (e.g., cells, e.g., nucleic acids) can include liquid nitrogen storage tanks and/or other freezer systems. Liquid nitrogen tanks provide temperature (e.g., about −195° C.) and/or humidity control, and can be used to store, for example, immortalized cell lines (e.g., immortalized iPSCs) over a long period of time. Alternatively, biological material (e.g., nucleic acids) can be stored in freezer systems at higher temperatures (e.g., from about −80° C. to about −20° C.). Additional equipment, backup systems, software/inventory control systems, sample location systems, automated sample retrieval, etc. can be used for storage and/or maintenance of the biological sample material stored in the repositories. The described setup allows for backup systems (e.g., additional repositories) to be used if a given tank and/or freezer temperature control system and/or humidity control system malfunctions.

Moreover, the provided systems and methods can record and track, via a graphical user interface, biological samples (and biological material extracted therefrom) used to generate genotyping data, for example, as described in U.S. Application No. 62/485,778, entitled “Chain Of Custody For Biological Samples And Biological Material Used In Genotyping Tests” and filed on Apr. 14, 2017, U.S. application Ser. No. 15/846,659 entitled “Chain Of Custody For Biological Samples And Biological Material Used In Genotyping Tests” filed on Dec. 19, 2017, and International Application No. PCT/US17/67272 entitled “Chain of Custody for Biological Samples and Biological Material Used in Genotyping Tests” filed on Dec. 19, 2017, the contents of which are hereby incorporated by reference in their entirety.

For example, as biological samples are processed in several stages to extract biological material and perform genotyping tests, IDs are assigned to biological sample material for individuals as well as well plates used during processing of the biological sample material in order to organize the samples and the tests. Biological sample materials are assigned to well plates for use in extracting biological material. Biological sample material is assigned to genotyping plates for use in performing genotyping tests. By associating IDs corresponding to biological sample material with IDs for well plates or genotyping plates, respectively, a user can track which extractions and/or tests need to be performed as well as record which biological samples have been received or genotyping plates analyzed via a graphical user interface.

### Illustrative Computer Network Environment

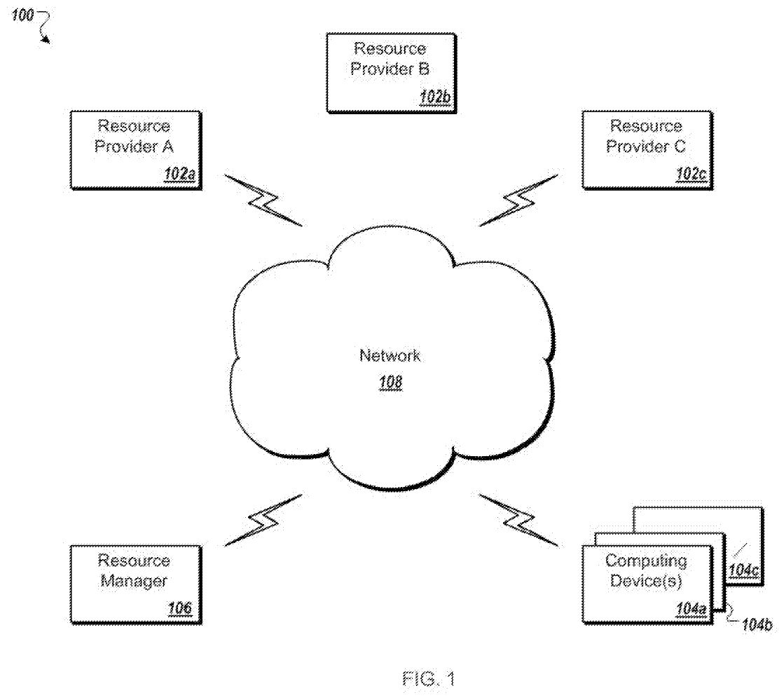

FIG. 1 shows an illustrative network environment 100 for use in the methods and systems described herein. In brief overview, referring now to FIG. 1, a block diagram of an exemplary cloud computing environment 100 is shown and described. The cloud computing environment 100 may include one or more resource providers 102a, 102b, 102c (collectively, 102). Each resource provider 102 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 102 may be connected to any other resource provider 102 in the cloud computing environment 100. In some implementations, the resource providers 102 may be connected over a computer network 108. Each resource provider 102 may be connected to one or more computing device 104a, 104b, 104c (collectively, 104), over the computer network 108.

The cloud computing environment 100 may include a resource manager 106. The resource manager 106 may be connected to the resource providers 102 and the computing devices 104 over the computer network 108. In some implementations, the resource manager 106 may facilitate the provision of computing resources by one or more resource providers 102 to one or more computing devices 104. The resource manager 106 may receive a request for a computing resource from a particular computing device 104. The resource manager 106 may identify one or more resource providers 102 capable of providing the computing resource requested by the computing device 104. The resource manager 106 may select a resource provider 102 to provide the computing resource. The resource manager 106 may facilitate a connection between the resource provider 102 and a particular computing device 104. In some implementations, the resource manager 106 may establish a connection between a particular resource provider 102 and a particular computing device 104. In some implementations, the resource manager 106 may redirect a particular computing device 104 to a particular resource provider 102 with the requested computing resource.

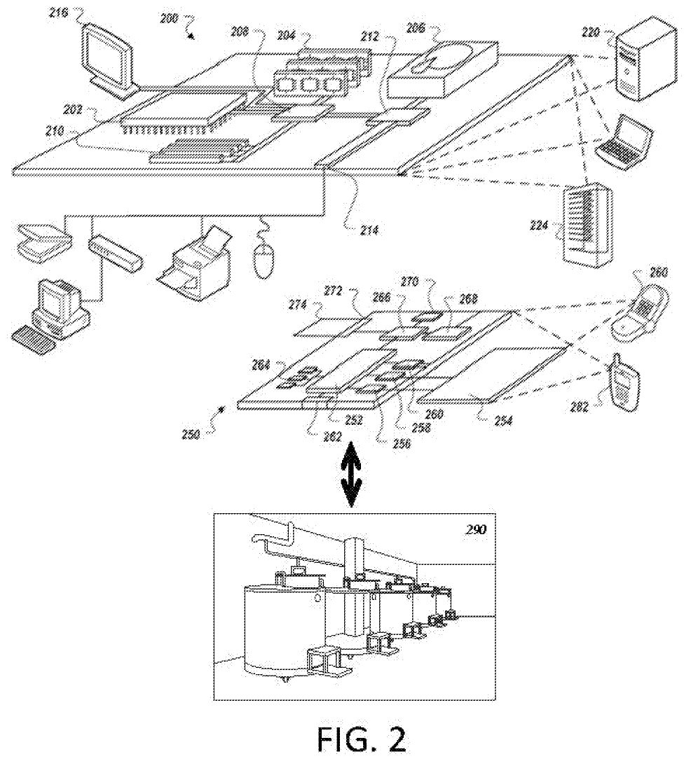

FIG. 2 shows an example of a computing device 200 and a mobile computing device 250 that can be used in the methods and systems described in this disclosure. The computing device 200 is intended to represent various forms of digital computers, such as laptops, desktops, workstations, personal digital assistants, servers, blade servers, mainframes, and other appropriate computers. The mobile computing device 250 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 200 includes a processor 202, a memory 204, a storage device 206, a high-speed interface 208 connecting to the memory 204 and multiple high-speed expansion ports 210, and a low-speed interface 212 connecting to a low-speed expansion port 214 and the storage device 206. Each of the processor 202, the memory 204, the storage device 206, the high-speed interface 208, the high-speed expansion ports 210, and the low-speed interface 212, are interconnected using various busses, and may be mounted on a common motherboard or in other manners as appropriate. The processor 202 can process instructions for execution within the computing device 200, including instructions stored in the memory 204 or on the storage device 206 to display graphical information for a GUI on an external input/output device, such as a display 216 coupled to the high-speed interface 208. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 204 stores information within the computing device 200. In some implementations, the memory 204 is a volatile memory unit or units. In some implementations, the memory 204 is a non-volatile memory unit or units. The memory 204 may also be another form of computer-readable medium, such as a magnetic or optical disk.

The storage device 206 is capable of providing mass storage for the computing device 200. In some implementations, the storage device 206 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 202), perform one or more methods, such as those described above. The instructions can also be stored by one or more storage devices such as computer- or machine-readable mediums (for example, the memory 204, the storage device 206, or memory on the processor 202).

The high-speed interface 208 manages bandwidth-intensive operations for the computing device 200, while the low-speed interface 212 manages lower bandwidth-intensive operations. Such allocation of functions is an example only. In some implementations, the high-speed interface 208 is coupled to the memory 204, the display 216 (e.g., through a graphics processor or accelerator), and to the high-speed expansion ports 210, which may accept various expansion cards (not shown). In the implementation, the low-speed interface 212 is coupled to the storage device 206 and the low-speed expansion port 214. The low-speed expansion port 214, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 200 may be implemented in a number of different forms, as shown in the figure. For example, it may be implemented as a standard server 220, or multiple times in a group of such servers. In addition, it may be implemented in a personal computer such as a laptop computer 222. It may also be implemented as part of a rack server system 224. Alternatively, components from the computing device 200 may be combined with other components in a mobile device (not shown), such as a mobile computing device 250. Each of such devices may contain one or more of the computing device 200 and the mobile computing device 250, and an entire system may be made up of multiple computing devices communicating with each other.

The mobile computing device 250 includes a processor 252, a memory 264, an input/output device such as a display 254, a communication interface 266, and a transceiver 268, among other components. The mobile computing device 250 may also be provided with a storage device, such as a micro-drive or other device, to provide additional storage. Each of the processor 252, the memory 264, the display 254, the communication interface 266, and the transceiver 268, are interconnected using various buses, and several of the components may be mounted on a common motherboard or in other manners as appropriate.

The processor 252 can execute instructions within the mobile computing device 250, including instructions stored in the memory 264. The processor 252 may be implemented as a chipset of chips that include separate and multiple analog and digital processors. The processor 252 may provide, for example, for coordination of the other components of the mobile computing device 250, such as control of user interfaces, applications run by the mobile computing device 250, and wireless communication by the mobile computing device 250.

The processor 252 may communicate with a user through a control interface 258 and a display interface 256 coupled to the display 254. The display 254 may be, for example, a TFT (Thin-Film-Transistor Liquid Crystal Display) display or an OLED (Organic Light Emitting Diode) display, or other appropriate display technology. The display interface 256 may comprise appropriate circuitry for driving the display 254 to present graphical and other information to a user. The control interface 258 may receive commands from a user and convert them for submission to the processor 252. In addition, an external interface 262 may provide communication with the processor 252, so as to enable near area communication of the mobile computing device 250 with other devices. The external interface 262 may provide, for example, for wired communication in some implementations, or for wireless communication in other implementations, and multiple interfaces may also be used.

The memory 264 stores information within the mobile computing device 250. The memory 264 can be implemented as one or more of a computer-readable medium or media, a volatile memory unit or units, or a non-volatile memory unit or units. An expansion memory 274 may also be provided and connected to the mobile computing device 250 through an expansion interface 272, which may include, for example, a SIMM (Single In Line Memory Module) card interface. The expansion memory 274 may provide extra storage space for the mobile computing device 250, or may also store applications or other information for the mobile computing device 250. Specifically, the expansion memory 274 may include instructions to carry out or supplement the processes described above, and may include secure information also. Thus, for example, the expansion memory 274 may be provided as a security module for the mobile computing device 250, and may be programmed with instructions that permit secure use of the mobile computing device 250. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 252), perform one or more methods, such as those described above. The instructions can also be stored by one or more storage devices, such as one or more computer- or machine-readable mediums (for example, the memory 264, the expansion memory 274, or memory on the processor 252). In some implementations, the instructions can be received in a propagated signal, for example, over the transceiver 268 or the external interface 262.

The mobile computing device 250 may communicate wirelessly through the communication interface 266, which may include digital signal processing circuitry where necessary. The communication interface 266 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 268 using a radio-frequency. In addition, short-range communication may occur, such as using a Bluetooth®, Wi-Fi™, or other such transceiver (not shown). In addition, a GPS (Global Positioning System) receiver module 270 may provide additional navigation- and location-related wireless data to the mobile computing device 250, which may be used as appropriate by applications running on the mobile computing device 250.

The mobile computing device 250 may also communicate audibly using an audio codec 260, which may receive spoken information from a user and convert it to usable digital information. The audio codec 260 may likewise generate audible sound for a user, such as through a speaker, e.g., in a handset of the mobile computing device 250. Such sound may include sound from voice telephone calls, may include recorded sound (e.g., voice messages, music files, etc.) and may also include sound generated by applications operating on the mobile computing device 250.

The mobile computing device 250 may be implemented in a number of different forms, as shown in the figure. For example, it may be implemented as a cellular telephone 280. It may also be implemented as part of a smart-phone 282, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In certain embodiments, the system comprises a physical biorepository 290 (comprising one or more cell storage containers) in communication with any of the computer system arrangements of FIG. 1 or 2.

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader and are not intended to be limiting with respect to the claimed subject matter.

Documents are incorporated herein by reference as noted. Where there is any discrepancy in the meaning of a particular term, the meaning provided in the Definition section above is controlling.

Certain embodiments of the present invention are described herein. It is, however, expressly noted that the present invention is not limited to these embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description. Therefore, the disclosure should not be limited to certain implementations, but rather should be limited only by the spirit and scope of the claims.

### EXEMPLIFICATION

### Example 1: Secretomes of Different Cell Types

Different types of cells secrete different organic and inorganic elements and molecules (e.g., proteins, DNA, exosomes, vesicles, etc.) into their environment. Tables 1-4 list the various proteins and the genes associated with these proteins that were identified in different cell types. Specifically, the secretomes of Retinal Pigment Epithelium (RPEs), Chondrocytes, Mesenchymal Stem Cells (MSCs), and Induced Pluripotent Stem Cells (iPSCs) were studied and analyzed. RPEs, Chondrocytes, and MSCs were differentiated from iPSCs. The secretomes of each of these cell types contain different proteins (e.g. cytokines). One and/or multiple proteins isolated from the secretome of a cell type may be used to derive a “personalized” cell-derived secretome composition and/or cytokine composition and may be used in the treatment of disease or as other therapy of a specific individual and/or group of individuals. Moreover, the “personalized” cell-derived composition may comprise the complete secretome or a subset of the secretome with the one or more desired cytokines suitable for cytokine therapy and/or exosomes for exosome therapy and/or microvesicles for microvesicle therapy of a specific individual and/or specific group of individuals.

TABLE 1
Retinal Pigment Epithelium (RPE) Secretome

| Protein | Gene | Description |
|---|---|---|
| Q4VY20 | YWHAB | 14-3-3 protein beta/alpha |
| Q04917 | YWHAH | 14-3-3 protein eta |
| P61981 | YWHAG | 14-3-3 protein gamma |
| Q6LD62 | 14-3-3 protein | 14-3-3 protein (cytosolic phospholipase A2 protein) |
| E5RIR4 | YWHAZ | 14-3-3 protein zeta/delta |
| H0YJ11 | ACTN1 | Alpha-actinin-1 |
| Q6GMP2 | ENO1 | Alpha-enolase |
| A0A0A0MRG2 | APP | Amyloid beta A4 protein |
| P13929 | ENO3 | Beta-enolase |
| E2DRY6 | | C-myc promoter-binding protein 1 |
| P12109 | COL6A1 | Collagen alpha-1(VI) chain |
| X5DNI1 | CRMP1 | Collapsin response mediator protein 1 isoform A |
| A0A024RDZ4 | DAOA | D-amino acid oxidase activator, isoform CRA_c |
| E9PD68 | CRMP1 | Dihydropyrimidinase-related protein 1 |
| Q5JR01 | EEF1A1 | Elongation factor 1-alpha 1 |
| Q05639 | EEF1A2 | Elongation factor 1-alpha 2 |
| Q92556 | ELMO1 | Engulfment and cell motility protein 1 |
| Q6FHV6 | ENO2 | ENO2 protein |
| A0A024R4F1 | ENO1 | Enolase 1, (Alpha), isoform CRA_a |
| D3DTL4 | ENO3 | Enolase 3 (Beta, muscle), isoform CRA_c |
| D0PNI1 | YWHAZ | Epididymis luminal protein 4 |
| V9HWD6 | HEL-S-1 | Epididymis secretory protein Li 1 |
| V9HWE9 | HEL-S-22 | Epididymis secretory protein Li 22 |
| V9HW41 | HEL-S-71 | Epididymis secretory protein Li 71 |
| V9HW12 | HEL-S-2a | Epididymis secretory sperm binding protein Li 2a |
| E9KL26 | SERPING1 | Epididymis tissue protein Li 173 |
| C9J4W5 | EIF5A2 | Eukaryotic translation initiation factor 5A |
| Q9GZV4 | EIF5A2 | Eukaryotic translation initiation factor 5A-2 |
| P09104 | ENO2 | Gamma-enolase |
| P09211 | GSTP1 | Glutathione S-transferase P |
| Q9HD26 | GOPC | Golgi-associated PDZ and coiled-coil motif-containing |
| | | protein |
| B4DV51 | RAN | GTP-binding nuclear protein Ran |
| P22626 | HNRNPA2B1 | Heterogeneous nuclear ribonucleoproteins A2/B1 |
| P09429 | HMGB1 | High mobility group protein B1 |
| A0A024RAS2 | H2AFJ | Histone H2A |
| A0A0U1RR32 | HIST1H3D | Histone H2A |
| A3KPC7 | HIST1H2AH | Histone H2A |
| A4FTV9 | HIST1H2AK | Histone H2A |
| Q96QV6 | HIST1H2AA | Histone H2A type 1-A |
| P04908 | HIST1H2AB | Histone H2A type 1-B/E |
| Q93077 | HIST1H2AC | Histone H2A type 1-C |
| P20671 | HIST1H2AD | Histone H2A type 1-D |
| Q96KK5 | HIST1H2AH | Histone H2A type 1-H |
| Q99878 | HIST1H2AJ | Histone H2A type 1-J |
| Q6FI13 | HIST2H2AA3 | Histone H2A type 2-A |
| Q8IUE6 | HIST2H2AB | Histone H2A type 2-B |
| Q16777 | HIST2H2AC | Histone H2A type 2-C |
| Q7L7L0 | HIST3H2A | Histone H2A type 3 |
| Q9BTM1 | H2AFJ | Histone H2A.J |
| Q71UI9 | H2AFV | Histone H2A.V |
| P0C0S5 | H2AFZ | Histone H2A.Z |
| P16104 | H2AFX | Histone H2AX |
| A0A024QZZ7 | HIST1H2BD | Histone H2B |
| A0A024RCJ2 | HIST1H2BJ | Histone H2B |
| A0A024RCJ9 | HIST1H2BN | Histone H2B |
| A0A024RCL8 | HIST1H2BK | Histone H2B |
| B2R4S9 | HIST1H2BI | Histone H2B |
| I6L9F7 | HIST1H2BM | Histone H2B |
| U3KQK0 | HIST1H2BN | Histone H2B |
| Q96A08 | HIST1H2BA | Histone H2B type 1-A |
| P33778 | HIST1H2BB | Histone H2B type 1-B |
| P62807 | HIST1H2BC | Histone H2B type 1-C/E/F/G/I |
| P58876 | HIST1H2BD | Histone H2B type 1-D |
| Q93079 | HIST1H2BH | Histone H2B type 1-H |
| P06899 | HIST1H2BJ | Histone H2B type 1-J |
| O60814 | HIST1H2BK | Histone H2B type 1-K |
| Q99880 | HIST1H2BL | Histone H2B type 1-L |
| Q99879 | HIST1H2BM | Histone H2B type 1-M |
| Q99877 | HIST1H2BN | Histone H2B type 1-N |
| P23527 | HIST1H2BO | Histone H2B type 1-O |
| Q16778 | HIST2H2BE | Histone H2B type 2-E |
| Q5QNW6 | HIST2H2BF | Histone H2B type 2-F |
| C9JMY1 | IGFBP2 | Insulin-like growth factor-binding protein 2 |
| P07195 | LDHB | L-lactate dehydrogenase B chain |
| Q8TE34 | SP100 | Nuclear autoantigen Sp-100 |
| B4DNG0 | OLFML3 | Olfactomedin-like protein 3 |
| P32119 | PRDX2 | Peroxiredoxin-2 |
| B4DHM5 | | Phosphoglycerate kinase |
| Q59EI5 | SERPING1 | Plasma protease C1 inhibitor |
| P07737 | PFN1 | Profilin-1 |
| Q5VTE0 | EEF1A1P5 | Putative elongation factor 1-alpha-like 3 |
| B2RPK0 | HMGB1P1 | Putative high mobility group protein B1-like 1 |
| Q9BSV4 | SFPQ | SFPQ protein |
| Q75MM1 | WUGSC:H_NH0244E06.1 | Similar to nonhistone chromosomal protein HMG-1 |
| | | [Homo sapiens] |
| Q86VG2 | SFPQ | Splicing factor proline/glutamine-rich (Polypyrimidine tract |
| | | binding protein associated) |
| Q9UKZ4 | TENM1 | Teneurin-1 |
| Q96HK4 | PRDX3 | Thioredoxin-dependent peroxide reductase, mitochondrial |
| Q96B85 | TUBB | TUBB protein |
| Q96HX0 | TUBB2C | TUBB2C protein |
| A0A024QZU2 | TUBB2B | Tubulin beta chain |
| A0A075B736 | TUBB8 | Tubulin beta chain |
| B3KS31 | TUBB6 | Tubulin beta chain |
| P07437 | TUBB | Tubulin beta chain |
| Q3ZCR3 | TUBB3 | Tubulin beta chain |
| Q8IZ29 | TUBB2C | Tubulin beta chain |
| Q9BUU9 | TUBB | Tubulin beta chain |
| Q9BV28 | TUBB3 | Tubulin beta chain |
| Q9UGA2 | DKFZp566F223 | Tubulin beta chain |
| Q9H4B7 | TUBB1 | Tubulin beta-1 chain |
| Q13885 | TUBB2A | Tubulin beta-2A chain |
| Q9BVA1 | TUBB2B | Tubulin beta-2B chain |
| Q13509 | TUBB3 | Tubulin beta-3 chain |
| P04350 | TUBB4A | Tubulin beta-4A chain |
| P68371 | TUBB4B | Tubulin beta-4B chain |
| Q9BUF5 | TUBB6 | Tubulin beta-6 chain |
| Q3ZCM7 | TUBB8 | Tubulin beta-8 chain |
| Q59EQ2 | | Tyrosine 3-monooxygenase/tryptophan 5-monooxygenase |
| | | activation protein, beta polypeptide variant |
| A0A024R1K7 | YWHAH | Tyrosine 3-monooxygenase/tryptophan 5-monooxygenase |
| | | activation protein, eta polypeptide, isoform CRA_b |
| F8VQQ8 | UBE2N | Ubiquitin-conjugating enzyme E2 N |
| B3KV61 | UXS1 | UDP-glucuronate decarboxylase 1, isoform CRA_a |
| C9JCB7 | UXS1 | UDP-glucuronic acid decarboxylase 1 |
| Q53TD0 | SP100 | Uncharacterized protein SP100 |
| A0AR27 | | Vacuolar protein sorting 45A isoform |
| A0A087WU65 | VPS45 | Vacuolar protein sorting-associated protein 45 |
| Q1KSF8 | XTP3TPATP1 | XTP3TPA-transactivated protein 1 |
| B3KML9 | | cDNA FLJ11352 fis, clone HEMBA1000020, highly similar |
| | | to Tubulin beta-2C chain |
| B3KNB4 | | CDNA FLJ14168 fis, clone NT2RP2001440, highly similar |
| | | to 14-3-3 protein gamma |
| B3KV96 | | cDNA FLJ16285 fis, clone OCBBF2004038, highly similar |
| | | to Dihydropyrimidinase-related protein 1 |
| B3KXQ5 | | cDNA FLJ45854 fis, clone OCBBF2024589, highly similar |
| | | to Dihydropyrimidinase-related protein 1 |
| B4DGD0 | | cDNA FLJ50491, highly similar to Amyloid beta A4 protein |
| | | (APP) (ABPP)(Alzheimer disease amyloid protein) (Cerebral |
| | | vascularamyloid peptide) (CVAP) (Protease nexin-II) (PN- |
| | | II)(APPI) (PreA4) |
| B7Z5D4 | | cDNA FLJ50613, highly similar to Vacuolar protein sorting- |
| | | associated protein 45 |
| B4DHC4 | | cDNA FLJ51843, highly similar to 14-3-3 protein gamma |
| B4DXZ5 | | cDNA FLJ52029, highly similar to Tubulin beta-7 chain |
| B4DE78 | | cDNA FLJ52141, highly similar to 14-3-3 protein gamma |
| B4E052 | | cDNA FLJ52378, highly similar to Tubulin beta-7 chain |
| B4DFH6 | | cDNA FLJ52536, highly similar to Tubulin beta-4 chain |
| B4DNE0 | | cDNA FLJ52573, highly similar to Elongation factor 1-alpha |
| | | 1 |
| B4E386 | | cDNA FLJ52847, highly similar to Tubulin beta-6 chain |
| B4DMU8 | | cDNA FLJ53063, highly similar to Tubulin beta-7 chain |
| B4DJ43 | | cDNA FLJ53341, highly similar to Tubulin beta-4 chain |
| B7Z4N1 | | cDNA FLJ53906, highly similar to Tubulin beta chain |
| B7Z360 | | cDNA FLJ54353, highly similar to Vacuolar protein sorting- |
| | | associated protein 45 |
| B4DM00 | | cDNA FLJ54367, highly similar to Amyloid beta A4 protein |
| | | (APP) (ABPP)(Alzheimer disease amyloid protein homolog) |
| B7Z2W3 | | cDNA FLJ54432, highly similar to Alpha-actinin-1 |
| B7Z5E4 | | cDNA FLJ54614, highly similar to Vacuolar protein sorting- |
| | | associated protein 45 |
| B7Z565 | | cDNA FLJ54739, highly similar to Alpha-actinin-1 |
| B4DJT9 | | cDNA FLJ59550, highly similar to Homo sapiens amyloid |
| | | beta (A4) protein, transcript variant 3, mRNA |
| A8K3B0 | | cDNA FLJ77877, highly similar to Human ENO2 neuron |
| | | specific (gamma) enolase |
| A8K3Q3 | | cDNA FLJ78230 |
| B2R6N6 | | cDNA, FLJ93036, highly similar to Homo sapiens tyrosine |
| | | 3-monooxy genase/tryptophan 5-monooxygenaseactivation |
| | | protein, eta polypeptide (YWHAH), mRNA |

TABLE 2
Chondrocytes Secretome

| Protein | Gene | Description |
|---|---|---|
| A0A024R6I7 | SERPINA1 | Alpha-1-antitrypsin |
| P61769 | B2M | Beta-2-microglobulin |
| A0A087WXW9 | COL5A1 | Collagen alpha-1(V) chain |
| P01024 | C3 | Complement C3 |
| A7L3I8 | ELN | Elastin |
| E9KL23 | SERPINA1 | Epididymis secretory sperm binding protein Li 44a |
| Q06828 | FMOD | Fibromodulin |
| H0Y4K8 | FN1 | Fibronectin |
| A6YID2 | FN1 | Fibronectin splice variant A |
| A6YID3 | FN1 | Fibronectin splice variant B |
| A6YID4 | FN1 | Fibronectin splice variant C |
| A6YID5 | FN1 | Fibronectin splice variant D |
| A6YID6 | FN1 | Fibronectin splice variant E |
| G3XA98 | FBLN5 | Fibulin 5, isoform CRA_a |
| Q9UBX5 | FBLN5 | Fibulin-5 |
| Q6PJE5 | FN1 | FN1 protein |
| K7EJY8 | LGALS3BP | Galectin-3-binding protein |
| C9JN98 | SERPINE2 | Glia-derived nexin |
| A0A024QZV0 | hCG_1811539 | HCG1811539, isoform CRA_b |
| A0A024QZZ7 | HIST1H2BD | Histone H2B |
| P33778 | HIST1H2BB | Histone H2B type 1-B |
| P62807 | HIST1H2BC | Histone H2B type 1-C/E/F/G/I |
| P58876 | HIST1H2BD | Histone H2B type 1-D |
| Q93079 | HIST1H2BH | Histone H2B type 1-H |
| P06899 | HIST1H2BJ | Histone H2B type 1-J |
| O60814 | HIST1H2BK | Histone H2B type 1-K |
| Q99880 | HIST1H2BL | Histone H2B type 1-L |
| Q99879 | HIST1H2BM | Histone H2B type 1-M |
| Q99877 | HIST1H2BN | Histone H2B type 1-N |
| P23527 | HIST1H2BO | Histone H2B type 1-O |
| Q16778 | HIST2H2BE | Histone H2B type 2-E |
| Q5QNW6 | HIST2H2BF | Histone H2B type 2-F |
| H0YKS8 | MFGE8 | Lactadherin |
| A0A0S2Z3Y1 | LGALS3BP | Lectin galactoside-binding soluble 3 binding protein isoform 1 |
| P51884 | LUM | Lumican |
| H0Y789 | TIMP1 | Metalloproteinase inhibitor 1 |
| A0A024RC59 | MFGE8 | Milk fat globule-EGF factor 8 protein, isoform CRA_b |
| P05121 | SERPINE1 | Plasminogen activator inhibitor 1 |
| Q59EE7 | | Pro-alpha-1 type V collagen variant |
| A0A024R8G3 | PTGDS | Prostaglandin D2 synthase 21 kDa (Brain), isoform CRA_a |
| D3DQH8 | SPARC | Secreted protein, acidic, cysteine-rich (Osteonectin), isoform |
| | | CRA_a |
| F8WCI6 | TF | Serotransferrin |
| A0A024R451 | SERPINE2 | Serpin peptidase inhibitor, clade E (Nexin, plasminogen activator |
| | | inhibitor type 1), member 2, isoform CRA_a |
| P09486 | SPARC | SPARC |
| D6RBY3 | SPON2 | Spondin-2 |
| O00391 | QSOX1 | Sulfhydryl oxidase 1 |
| Q8NBS9 | TXNDC5 | Thioredoxin domain-containing protein 5 |
| Q6FGX5 | TIMP1 | TIMP metallopeptidase inhibitor 1, isoform CRA_a |
| H0Y8L3 | TGFBI | Transforming growth factor-beta-induced protein ig-h3 |
| Q86UY0 | TXNDC5 | TXNDC5 protein |
| Q658S9 | DKFZp666I134 | Uncharacterized protein DKFZp666I134 |
| Q5CZ99 | DKFZp686I1370 | Uncharacterized protein DKFZp686I1370 |
| Q6N084 | DKFZp686L11144 | Uncharacterized protein DKFZp686L11144 |
| Q6MZM7 | DKFZp686O12165 | Uncharacterized protein DKFZp686O12165 |
| Q68CX6 | DKFZp686O13149 | Uncharacterized protein DKFZp686O13149 |
| B3KP88 | | cDNA FLJ31415 fis, clone NT2NE2000284, highly similar to |
| | | Galectin-3-binding protein |
| B3KTQ2 | | cDNA FLJ38589 fis, clone HCHON2010074, highly similar to |
| | | LACTADHERIN |
| Q6ZUN2 | | cDNA FLJ43523 fis, clone PLACE5000282, weakly similar to |
| | | Homo sapiens elastin (supravalvular aortic stenosis, Williams- |
| | | Beuren syndrome) (ELN) |
| B4DTK1 | | cDNA FLJ53292, highly similar to Homo sapiens fibronectin 1 |
| | | (FN1), transcript variant 5, mRNA |
| B4DN21 | | cDNA FLJ53365, highly similar to Homo sapiens fibronectin 1 |
| | | (FN1), transcript variant 4, mRNA |
| B4DVE1 | | cDNA FLJ53478, highly similar to Galectin-3-binding protein |
| B4E1J3 | | cDNA FLJ53615, highly similar to Fibromodulin |
| B4DU16 | | cDNA FLJ54550, highly similar to Homo sapiens fibronectin 1 |
| | | (FN1), transcript variant 6, mRNA |
| B4DDG4 | | cDNA FLJ54583, highly similar to Galectin-3-binding protein |
| B4DRV4 | | cDNA FLJ55667, highly similar to Secreted protein acidic and |
| | | rich in cysteine |
| B4E3S4 | | cDNA FLJ56005, highly similar to Elastin |
| B4E396 | | cDNA FLJ59612, highly similar to Lactadherin |
| B3KQA8 | | cDNA FLJ90059 fis, clone HEMBA1003230, highly similar to |
| | | Fibulin-5 |
| B3KQF4 | | cDNA FLJ90373 fis, clone NT2RP2004606, highly similar to |
| | | Metalloproteinase inhibitor 1 |
| Q8NBI4 | | cDNA PSEC0254 fis, clone NT2RP3003474, moderately similar |
| | | to ELASTIN |
| B7ZAB0 | | cDNA, FLJ79124, highly similar to Plasminogen activator |
| | | inhibitor 1 |
| B2RDL6 | | cDNA, FLJ96669, highly similar to Homo sapiens secreted |
| | | protein, acidic, cysteine-rich (osteonectin)(SPARC), mRNA |
| Q86TV4 | | Full-length cDNA clone CS0DI085YI08 of Placenta of |
| | | Homo sapiens (human) |

TABLE 3
Mesenchymal Stem Cell (MSC) Secretome

| Protein | Gene | Description |
|---|---|---|
| P08253 | MMP2 | 72 kDa type IV collagenase |
| D3GKD8 | HBG1 | A-gamma globin Osilo variant |
| Q14474 | | A-gamma-hemoglobin gene from Greek HPFH mutant |
| Q562L3 | ACT | Actin-like protein |
| O00468 | AGRN | Agrin |
| Q86YQ4 | HBA1 | Alpha-1 globin |
| H0YNP5 | ANXA2 | Annexin |
| G1FM86 | | Anti-Influenza A hemagglutinin heavy chain variable region |
| A2JA18 | | Anti-mucin1 heavy chain variable region |
| F5H6I0 | B2M | Beta-2-microglobulin |
| H0YLF3 | B2M | Beta-2-microglobulin |
| P61769 | B2M | Beta-2-microglobulin |
| E5RG95 | ENO3 | Beta-enolase |
| E5RGZ4 | ENO3 | Beta-enolase |
| E5RI09 | ENO3 | Beta-enolase |
| K7EPM1 | ENO3 | Beta-enolase |
| P13929 | ENO3 | Beta-enolase |
| Q9UM85 | beta-globin | Beta-globin protein |
| F2RM37 | F9 p22 | Coagulation factor IX |
| Q9UML6 | COL1A1 | Collagen alpha-1(I) chain |
| A0A087WXW9 | COL5A1 | Collagen alpha-1(V) chain |
| P12109 | COL6A1 | Collagen alpha-1(VI) chain |
| Q6LAN8 | COL1A1 | Collagen type I alpha 1 |
| A0A0S2Z3K0 | COL1A2 | Collagen type I alpha 2 isoform 5 |
| B1N7B6 | | Cryocrystalglobulin CC1 heavy chain variable region |
| Q8IUB0 | | CTCL tumor antigen HD-CL-08 |
| A0A0K0K1J1 | HEL-S-2 | Cystatin |
| Q9UJU1 | VIL2 | Cytovillin 2 |
| Q9UJZ2 | VIL2 | Cytovillin 2 |
| F8VX58 | DCN | Decorin |
| Q504Z0 | EEF1A1 | EEF1A1 protein |
| Q6P082 | EEF1A1 | EEF1A1 protein |
| Q16577 | PTI-1 | Elongation factor 1-alpha 1 |
| Q6FHV6 | ENO2 | ENO2 protein |
| D3DTL4 | ENO3 | Enolase 3 (Beta, muscle), isoform CRA_c |
| V9HWC0 | HEL70 | Epididymis luminal protein 70 |
| V9HW42 | HEL-S-105 | Epididymis secretory protein Li 105 |
| V9HWE9 | HEL-S-22 | Epididymis secretory protein Li 22 |
| Q86Z22 | HEL-S-297 | Epididymis secretory protein Li 297 |
| V9HWC7 | HEL-S-128m | Epididymis secretory sperm binding protein Li 128m |
| Q53HR1 | | Eukaryotic translation elongation factor 1 alpha 1 variant |
| Q59GP5 | | Eukaryotic translation elongation factor 1 alpha 2 variant |
| C9J4W5 | EIF5A2 | Eukaryotic translation initiation factor 5A |
| Q6IS14 | EIF5AL1 | Eukaryotic translation initiation factor 5A-1-like |
| Q9GZV4 | EIF5A2 | Eukaryotic translation initiation factor 5A-2 |
| A0A087WWR8 | SULF1 | Extracellular sulfatase Sulf-1 |
| E7EQR4 | EZR | Ezrin |
| E7CYP2 | HBG2 | G gamma globin chain |
| D3GKD9 | HBG2 | G-gamma globin Paulinia variant |
| Q14476 | | G-gamma-hemoglobin gene from Greek HPFH mutant |
| P09104 | ENO2 | Gamma-enolase |
| B7UCU6 | HBG2 | Gamma-globin chain |
| A0A109PVK5 | | GCT-A6 heavy chain variable region |
| A0A125QYY4 | | GCT-A7 heavy chain variable region |
| D9YZU8 | HBG1 | Globin B1 |
| D9YZU9 | HBG2 | Globin B2 |
| D9YZU7 | HBE1 | Globin B3 |
| A0A1W6AYU6 | | Glucose-6-phosphate isomerase |
| P09211 | GSTP1 | Glutathione S-transferase P |
| E5FY30 | | Glycoprotein Ib |
| A0A0C4DGZ8 | GP1BA | Glycoprotein Ib (Platelet), alpha polypeptide |
| A2NYV1 | | Heavy chain Fab |
| Q86YQ1 | HBA2 | Hemoglobin alpha-2 |
| Q9UNL6 | HBG2 | Hemoglobin gamma-G |
| A8MUF7 | HBE1 | Hemoglobin subunit epsilon |
| P69891 | HBG1 | Hemoglobin subunit gamma-1 |
| E9PBW4 | HBG2 | Hemoglobin subunit gamma-2 |
| Q99729 | HNRNPAB | Heterogeneous nuclear ribonucleoprotein A/B |
| A0A024RA28 | HNRPA2B1 | Heterogeneous nuclear ribonucleoprotein A2/B1, isoform CRA_d |
| Q5T7C4 | HMGB1 | High mobility group protein B1 |
| A0A068LN07 | | Ig heavy chain variable region |
| A0A087WSY4 | IGHV4-30-2 | Immunoglobulin heavy variable 4-30-2 |
| A0A075B6R2 | IGHV4-4 | Immunoglobulin heavy variable 4-4 |
| A0A075B7B6 | IGHV4OR15-8 | Immunoglobulin heavy variable 4/OR15-8 (non-functional) |
| A0A0J9YXX1 | IGHV5-10-1 | Immunoglobulin heavy variable 5-10-1 |
| A0A0C4DH38 | IGHV5-51 | Immunoglobulin heavy variable 5-51 |
| A0A024R433 | IGFBP5 | Insulin-like growth factor binding protein 5, isoform CRA_a |
| A0A024R6R4 | MMP2 | Matrix metallopeptidase 2 (Gelatinase A, 72 kDa gelatinase, 72 kDa |
| | | type IV collagenase), isoform CRA_a |
| G8JLP4 | MARF1 | Meiosis regulator and mRNA stability factor 1 |
| P26038 | MSN | Moesin |
| A0A109PS45 | | MS-D1 heavy chain variable region |
| Q6PJT4 | MSN | MSN protein |
| A0A193AUJ5 | GP1BA | Mutant platelet membrane glycoprotein Ib-alpha |
| Q9UL73 | | Myosin-reactive immunoglobulin heavy chain variable region |
| P23284 | PPIB | Peptidyl-prolyl cis-trans isomerase B |
| A0A024RDS2 | POSTN | Periostin, osteoblast specific factor, isoform CRA_c |
| P30041 | PRDX6 | Peroxiredoxin-6 |
| V9HW85 | HEL-S-272 | Phosphoglycerate kinase |
| P07205 | PGK2 | Phosphoglycerate kinase 2 |
| A5CKE2 | GP1BA | Platelet glycoprotein Ib alpha |
| Q59EE7 | | Pro-alpha-1 type V collagen variant |
| Q15113 | PCOLCE | Procollagen C-endopeptidase enhancer 1 |
| P00491 | PNP | Purine nucleoside phosphorylase |
| Q8N7G1 | | Purine nucleoside phosphorylase |
| B2RPK0 | HMGB1P1 | Putative high mobility group protein B1-like 1 |
| B0YJ88 | RDX | Radixin |
| A2J1N5 | | Rheumatoid factor RF-ET6 |
| A2J1M8 | | Rheumatoid factor RF-IP12 |
| D3DQH8 | SPARC | Secreted protein, acidic, cysteine-rich (Osteonectin), isoform |
| | | CRA_a |
| F2RM35 | factor IX F9 | Serine protease |
| P02743 | APCS | Serum amyloid P-component |
| A0A0C4DGN2 | SHBG | Sex hormone-binding globulin |
| Q9H299 | SH3BGRL3 | SH3 domain-binding glutamic acid-rich-like protein 3 |
| Q65ZC8 | scFv | Single-chain Fv |
| P09486 | SPARC | SPARC |
| A0A024R809 | SULF1 | Sulfatase 1, isoform CRA_a |
| A0A140VJC8 | | Testicular tissue protein Li 2 |
| H0Y8L3 | TGFBI | Transforming growth factor-beta-induced protein ig-h3 |
| Q53GU8 | | Transforming growth factor, beta-induced, 68 kDa variant |
| Q96RE1 | EEF1A1L14 | Translation elongation factor 1 alpha 1-like 14 |
| Q19UH6 | F9 | Truncated coagulation factor IX |
| Q9H4B7 | TUBB1 | Tubulin beta-1 chain |
| Q13509 | TUBB3 | Tubulin beta-3 chain |
| J7M2B1 | EZR-ROS1 | Tyrosine-protein kinase receptor |
| Q75N18 | COL1A2 | Uncharacterized protein COL1A2 |
| Q7Z2W2 | DKFZp686F13142 | Uncharacterized protein DKFZp686F13142 |
| B3KQ05 | | CDNAFLJ32558 fis, clone SPLEN1000143, highly similar to |
| | | High mobility group protein B1 |
| B7Z437 | | cDNA FLJ53435, highly similar to Ezrin |
| B4E2C5 | | cDNA FLJ54032, highly similar to Elongation factor 1-alpha 1 |
| B7Z5V2 | | cDNA FLJ54141, highly similar to Ezrin |
| B4DRV4 | | cDNA FLJ55667, highly similar to Secreted protein acidic and |
| | | rich in cysteine |
| B4DN66 | | cDNA FLJ56576, highly similar to Collagen alpha-2(I) chain |
| B4DMY3 | | cDNA FLJ60713, highly similar to Homo sapiens heterogeneous |
| | | nuclear ribonucleoprotein A/B (HNRPAB), transcript variant 1, |
| | | mRNA |
| A8K3B0 | | cDNA FLJ77877, highly similar to Human ENO2 neuron specific |
| | | (gamma) enolase |
| B2R6J2 | | cDNA, FLJ92973, highly similar to Homo sapiens villin 2 (ezrin) |
| | | (VIL2), mRNA |
| B2RDL6 | | cDNA, FLJ96669, highly similar to Homo sapiens secreted |
| | | protein, acidic, cysteine-rich (osteonectin)(SPARC), mRNA |

TABLE 4
Induced Pluripotent Stem Cell (iPSC) Secretome

| Protein | Gene | Description |
|---|---|---|
| A0AUL6 | ACTB | ACTB protein |
| Q96FU6 | ACTG1 | ACTG1 protein |
| Q7Z7J6 | ACTA1 | Actin alpha 1 skeletal muscle protein |
| Q562Z7 | ACT | Actin-like protein |
| P68032 | ACTC1 | Actin, alpha cardiac muscle 1 |
| P68133 | ACTA1 | Actin, alpha skeletal muscle |
| F6QUT6 | ACTA2 | Actin, aortic smooth muscle |
| G5E9R0 | ACTB | Actin, cytoplasmic 1 |
| P63261 | ACTG1 | Actin, cytoplasmic 2 |
| B8ZZJ2 | ACTG2 | Actin, gamma-enteric smooth muscle |
| P12814 | ACTN1 | Alpha-actinin-1 |
| K7EM90 | ENO1 | Alpha-enolase |
| V9HVU7 | CLEC2D | Alternative protein CLEC2D |
| J9ZVQ3 | APOE | Apolipoprotein E |
| A0A0S2Z3V0 | APOE | Apolipoprotein E isoform 2 |
| A0A0S2Z3B1 | APOE | Apolipoprotein E isoform 4 |
| Q0QEN7 | ATP5B | ATP synthase subunit beta |
| F1BXA6 | | Beta-actin |
| E2DRY6 | | C-myc promoter-binding protein 1 |
| A0A024RAP1 | CLEC2D | C-type lectin domain family 2, member D, isoform CRA_b |
| O15078 | CEP290 | Centrosomal protein of 290 kDa |
| Q8IUB0 | | CTCL tumor antigen HD-CL-08 |
| P04080 | CSTB | Cystatin-B |
| Q9UJU1 | VIL2 | Cytovillin 2 |
| Q6P4C9 | EEF1A1 | EEF1A1 protein |
| A0A087WV01 | EEF1A1 | Elongation factor 1-alpha |
| A0A024R4F1 | ENO1 | Enolase 1, (Alpha), isoform CRA_a |
| V9HWC0 | HEL70 | Epididymis luminal protein 70 |
| K9JA46 | EL52 | Epididymis luminal secretory protein 52 |
| V9HW42 | HEL-S-105 | Epididymis secretory protein Li 105 |
| V9HWE9 | HEL-S-22 | Epididymis secretory protein Li 22 |
| V9HW12 | HEL-S-2a | Epididymis secretory sperm binding protein Li 2a |
| V9HW63 | HEL-S-97n | Epididymis secretory sperm binding protein Li 97n |
| Q53HR1 | | Eukaryotic translation elongation factor 1 alpha 1 variant |
| Q59GP5 | | Eukaryotic translation elongation factor 1 alpha 2 variant |
| C9J4W5 | EIF5A2 | Eukaryotic translation initiation factor 5A |
| Q6IS14 | EIF5AL1 | Eukaryotic translation initiation factor 5A-1-like |
| Q9GZV4 | EIF5A2 | Eukaryotic translation initiation factor 5A-2 |
| E7EQR4 | EZR | Ezrin |
| E9PEB5 | FUBP1 | Far upstream element-binding protein 1 |
| Q96I24 | FUBP3 | Far upstream element-binding protein 3 |
| B4DUV1 | | Fibulin-1 |
| H3BQN4 | ALDOA | Fructose-bisphosphate aldolase |
| Q6PJY1 | FUBP1 | FUBP1 protein |
| Q86U12 | | Full-length cDNA clone CS0CAP007YF18 of Thymus of |
| | | Homo sapiens (human) |
| A0A087X243 | GSTP1 | Glutathione S-transferase P |
| Q0QET7 | GAPDH | Glyceraldehyde-3-phosphate dehydrogenase |
| V9HVZ4 | HEL-S-162eP | Glyceraldehyde-3-phosphate dehydrogenase |
| Q1KLZ0 | PS1TP5BP1 | HCG15971, isoform CRA_a |
| B4DMJ7 | hCG_2015269 | HCG2015269, isoform CRA_c |
| A0A024R8A7 | hCG_31253 | HCG31253, isoform CRA_a |
| A0A024RD80 | HSP90AB1 | Heat shock protein 90 kDa alpha (Cytosolic), class B member 1, |
| | | isoform CRA_a |
| P07900 | HSP90AA1 | Heat shock protein HSP 90-alpha |
| A0A024RA28 | HNRPA2B1 | Heterogeneous nuclear ribonucleoprotein A2/B1, isoform CRA_d |
| A0A024RDB4 | HNRPD | Heterogeneous nuclear ribonucleoprotein D (AU-rich element |
| | | RNA binding protein 1, 37 kDa), isoform CRA_c |
| Q5EC54 | HNRPK | Heterogeneous nuclear ribonucleoprotein K transcript variant |
| A0A024R228 | HNRPK | Heterogeneous nuclear ribonucleoprotein K, isoform CRA_d |
| A0A087WUI2 | HNRNPA2B1 | Heterogeneous nuclear ribonucleoproteins A2/B1 |
| P22626 | HNRNPA2B1 | Heterogeneous nuclear ribonucleoproteins A2/B1 |
| P09429 | HMGB1 | High mobility group protein B1 |
| A3R0T8 | HIST1H1E | Histone 1, H1e |
| Q4VB24 | HIST1H1E | Histone cluster 1, H1e |
| P16403 | HIST1H1C | Histone H1.2 |
| P16402 | HIST1H1D | Histone H1.3 |
| P10412 | HIST1H1E | Histone H1.4 |
| A0A024QZZ7 | HIST1H2BD | Histone H2B |
| Q96A08 | HIST1H2BA | Histone H2B type 1-A |
| P33778 | HIST1H2BB | Histone H2B type 1-B |
| P62807 | HIST1H2BC | Histone H2B type 1-C/E/F/G/I |
| P58876 | HIST1H2BD | Histone H2B type 1-D |
| Q93079 | HIST1H2BH | Histone H2B type 1-H |
| P06899 | HIST1H2BJ | Histone H2B type 1-J |
| O60814 | HIST1H2BK | Histone H2B type 1-K |
| Q99880 | HIST1H2BL | Histone H2B type 1-L |
| Q99879 | HIST1H2BM | Histone H2B type 1-M |
| Q99877 | HIST1H2BN | Histone H2B type 1-N |
| P23527 | HIST1H2BO | Histone H2B type 1-O |
| Q16778 | HIST2H2BE | Histone H2B type 2-E |
| Q5QNW6 | HIST2H2BF | Histone H2B type 2-F |
| P62805 | HIST1H4A | Histone H4 |
| Q2VPJ6 | HSP90AA1 | HSP90AA1 protein |
| H6VRG3 | KRT1 | Keratin 1 |
| P35527 | KRT9 | Keratin, type I cytoskeletal 9 |
| P04264 | KRT1 | Keratin, type II cytoskeletal 1 |
| V9HWB9 | HEL-S-133P | L-lactate dehydrogenase |
| F5GYU2 | LDHA | L-lactate dehydrogenase A chain |
| P07195 | LDHB | L-lactate dehydrogenase B chain |
| H0YKS8 | MFGE8 | Lactadherin |
| A3R0T7 | | Liver histone H1e |
| A0A024RC55 | MFGE8 | Milk fat globule-EGF factor 8 protein, isoform CRA_a |
| A0A024RC59 | MFGE8 | Milk fat globule-EGF factor 8 protein, isoform CRA_b |
| P26038 | MSN | Moesin |
| Q6PJT4 | MSN | MSN protein |
| Q9BTI9 | NPM1 | NPM1 protein |
| Q8WTW5 | NPM1 | Nucleophosmin |
| A0A0S2Z491 | NPM1 | Nucleophosmin isoform 2 |
| O75475 | PSIP1 | PC4 and SFRS1-interacting protein |
| A0A0A0MSI0 | PRDX1 | Peroxiredoxin-1 |
| P32119 | PRDX2 | Peroxiredoxin-2 |
| Q13162 | PRDX4 | Peroxiredoxin-4 |
| Q0D2Q6 | PGAM1 | Phosphoglycerate mutase |
| P15259 | PGAM2 | Phosphoglycerate mutase 2 |
| Q8N0Y7 | PGAM4 | Probable phosphoglycerate mutase 4 |
| P07737 | PFN1 | Profilin-1 |
| Q6PNN2 | | Prostate cancer antigen T21 |
| G3V5M2 | PNP | Purine nucleoside phosphorylase |
| Q5VTE0 | EEF1A1P5 | Putative elongation factor 1-alpha-like 3 |
| B2RPK0 | HMGB1P1 | Putative high mobility group protein B1-like 1 |
| Q6DN03 | HIST2H2BC | Putative histone H2B type 2-C |
| B0YJ88 | RDX | Radixin |
| Q6PKD3 | RDX | RDX protein |
| P49903 | SEPHS1 | Selenide, water dikinase 1 |
| Q9BSV4 | SFPQ | SFPQ protein |
| B8ZZJ0 | SUMO1 | Small ubiquitin-related modifier 1 |
| B8ZZ67 | SUMO1 | SMT3 suppressor of mif two 3 homolog 1 (Yeast), isoform |
| | | CRA_b |
| Q01082 | SPTBN1 | Spectrin beta chain, non-erythrocytic 1 |
| Q86VG2 | SFPQ | Splicing factor proline/glutamine-rich (Polypyrimidine tract |
| | | binding protein associated) |
| A0A140VKE5 | QSCN6 | Sulfhydryl oxidase |
| O00391 | QSOX1 | Sulfhydryl oxidase 1 |
| A0A140VJQ2 | | Testicular tissue protein Li 128 |
| Q96HK4 | PRDX3 | Thioredoxin-dependent peroxide reductase, mitochondrial |
| Q8NEM7 | SUPT20H | Transcription factor SPT20 homolog |
| Q96RE1 | EEF1A1L14 | Translation elongation factor 1 alpha 1-like 14 |
| A0A024QZU2 | TUBB2B | Tubulin beta chain |
| A5D906 | TUBB2A | Tubulin beta chain |
| G3V2A3 | TUBB3 | Tubulin beta chain |
| P07437 | TUBB | Tubulin beta chain |
| Q3ZCR3 | TUBB3 | Tubulin beta chain |
| Q8IZ29 | TUBB2C | Tubulin beta chain |
| Q9BVA1 | TUBB2B | Tubulin beta-2B chain |
| Q13509 | TUBB3 | Tubulin beta-3 chain |
| P68371 | TUBB4B | Tubulin beta-4B chain |
| J7M2B1 | EZR-ROS1 | Tyrosine-protein kinase receptor |
| Q96FW1 | OTUB1 | Ubiquitin thioesterase OTUB1 |
| Q9NX34 | | cDNA FLJ20465 fis, clone KAT06236 |
| B3KQ05 | | CDNAFLJ32558 fis, clone SPLEN1000143, highly similar to |
| | | High mobility group protein B1 |
| B3KTQ2 | | cDNA FLJ38589 fis, clone HCHON2010074, highly similar to |
| | | LACTADHERIN |
| B3KUD3 | | cDNA FLJ39583 fis, clone SKMUS2004897, highly similar to |
| | | ACTIN, ALPHA SKELETAL MUSCLE |
| B3KW67 | | cDNA FLJ42347 fis, clone UTERU2003399, highly similar to |
| | | Actin, gamma-enteric smooth muscle |
| B3KWQ3 | | cDNA FLJ43573 fis, clone RECTM2001691, highly similar to |
| | | Actin, cytoplasmic 2 |
| B4DKL5 | | cDNA FLJ51983, highly similar to Phosphoglycerate mutase 1 |
| | | (EC 5.4.2.1) |
| B4DJI1 | | cDNA FLJ52549, highly similar to L-lactate dehydrogenase A |
| | | chain (EC 1.1.1.27) |
| B4DNE0 | | cDNA FLJ52573, highly similar to Elongation factor 1-alpha 1 |
| B7Z6I1 | | cDNA FLJ52755, highly similar to Actin, aortic smooth muscle |
| B4DUI8 | | cDNA FLJ52761, highly similar to Actin, aortic smooth muscle |
| B4E335 | | cDNA FLJ52842, highly similar to Actin, cytoplasmic 1 |
| B4DMJ5 | | cDNA FLJ53012, highly similar to Tubulin beta-7 chain |
| B4DJA4 | | cDNA FLJ53048, highly similar to Phosphoglycerate mutase 1 |
| | | (EC 5.4.2.1) |
| B4DT31 | | cDNA FLJ53425, highly similar to Far upstream element-binding |
| | | protein 1 |
| B7Z437 | | cDNA FLJ53435, highly similar to Ezrin |
| B4DGL0 | | cDNA FLJ53619, highly similar to Heat shock protein HSP 90- |
| | | beta |
| B7Z6P1 | | cDNA FLJ53662, highly similar to Actin, alpha skeletal muscle |
| B4DMA2 | | cDNA FLJ54023, highly similar to Heat shock protein HSP 90- |
| | | beta |
| B4E2C5 | | cDNA FLJ54032, highly similar to Elongation factor 1-alpha 1 |
| B7Z5V2 | | cDNA FLJ54141, highly similar to Ezrin |
| B4DTC3 | | cDNA FLJ54150, highly similar to Heterogeneous nuclear |
| | | ribonucleoprotein D0 |
| B7Z2W3 | | cDNA FLJ54432, highly similar to Alpha-actinin-1 |
| B4DUQ1 | | cDNA FLJ54552, highly similar to Heterogeneous nuclear |
| | | ribonucleoprotein K |
| B7Z565 | | cDNA FLJ54739, highly similar to Alpha-actinin-1 |
| B4DW52 | | cDNA FLJ55253, highly similar to Actin, cytoplasmic 1 |
| B4E3A4 | | cDNA FLJ57283, highly similar to Actin, cytoplasmic 2 |
| B4DVQ0 | | cDNA FLJ58286, highly similar to Actin, cytoplasmic 2 |
| B4DWL1 | | cDNA FLJ59240, highly similar to Far upstream element-binding |
| | | protein 1 |
| B4E396 | | cDNA FLJ59612, highly similar to Lactadherin |
| B4DTA2 | | cDNA FLJ60148, highly similar to Homo sapiens heterogeneous |
| | | nuclear ribonucleoprotein D-like (HNRPDL), transcript variant 2, |
| | | mRNA |
| B4E0X8 | | cDNA FLJ61021, highly similar to Far upstream element-binding |
| | | protein 1 |
| A8K3W9 | | cDNA FLJ77842 |
| A8K3K1 | | cDNA FLJ78096, highly similar to Homo sapiens actin, alpha, |
| | | cardiac muscle (ACTC), mRNA |
| B7Z9E5 | | cDNA, FLJ78809, highly similar to Phosphoglycerate mutase 1 |
| | | (EC 5.4.2.1) |
| B7ZAP6 | | cDNA, FLJ79260, highly similar to Actin, cytoplasmic 2 |
| B2R6J2 | | cDNA, FLJ92973, highly similar to Homo sapiens villin 2 (ezrin) |
| | | (VIL2), mRNA |

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of manufacturing an induced pluripotent stem cell (iPSC)-derived secretome composition tailored for treatment of a particular subject or particular group of subjects, said method comprising the steps of:
(a) identifying, as compatible with the particular subject or particular group of subjects, one or more iPSCs and/or one or more iPSC-derived cells;
(b) retrieving compatible cells corresponding to the one or more iPSCs and/or one or more iPSC-derived cells identified as compatible with the particular subject or particular group of subjects; and
(c) producing the iPSC-derived secretome composition using the retrieved compatible cells.

2. The method of claim 1, wherein the one or more iPSCs and/or the one or more iPSC-derived cells are human cells.

3. The method of claim 1 or claim 2, wherein the iPSC-derived secretome composition comprises one or more desired compatible-cell-secreted species.

4. The method of claim 3, wherein the one or more desired compatible-cell-secreted species comprise one or more desired compatible-cell-secreted molecules and/or one or more desired compatible-cell-secreted biological elements.

5. The method of claim 3, wherein the one or more desired compatible-cell-secreted species comprise one or more cytokines.

6. The method of claim 3, wherein the one or more desired compatible-cell-secreted species comprise one or more exosomes and/or one or more microvesicles.

7. The method of any one of the preceding claims, wherein step (c) comprises extracting one or more desired compatible-cell-secreted molecules and/or one or more desired biological elements from the retrieved compatible cells.

8. The method of any one of the preceding claims, wherein step (b) comprises deriving the compatible cells from a biological sample of the particular subject.

9. The method of any one of the preceding claims, wherein step (c) comprises producing a lyophilized iPSC-derived secretome composition.

10. The method of any one of the preceding claims, wherein the retrieved compatible cells comprise one or more members selected from the group consisting of induced pluripotent stem cells (iPSCs), mesenchymal stem cells (MSCs), Retinal Pigment Epithelium (RPEs), chondrocytes, hematopoietic stem cells (HSCs), blood progenitor cells, and embryoid bodies.

11. The method of any one of the preceding claims, wherein the particular subject or the particular group of subjects is/are human.

12. The method of any one of the preceding claims, wherein the one or more iPSCs and/or one or more iPSC-derived cells are stored in a physical repository.

13. The method of any one of the preceding claims, wherein step (b) comprises obtaining the compatible cells from a physical repository.

14. The method of any one of the preceding claims, wherein step (b) comprises retrieving, by a processor of a computing device, one or more data entries corresponding to the compatible cells using a processor-based query from a user, wherein the query comprises an identification of a cell type indicative of compatibility with the particular subject or particular group of subjects.

15. The method of claim 14, wherein the identification of cell type indicative of compatibility with the particular subject or particular group of subjects comprises one or more of (i) to (iii): (i) an HLA match, (ii) an ABO blood type match, and (iii) an RHD blood group match.

16. The method of any one of the preceding claims, wherein the iPSC-derived secretome composition comprises the retrieved compatible cells.

17. The method of any one of the preceding claims, wherein step (c) comprises forming the retrieved compatible cells into a macroscopic structure suitable for topical application to the subject.

18. The method of claim 16, wherein the macroscopic structure is a sheet.

19. The method of any one of the preceding claims, wherein producing the iPSC-derived secretome composition in step (c) comprises exposing the compatible cells to culture media.

20. The method of claim 19, wherein the iPSC-derived secretome composition comprises the compatible cells, the culture media, and the one or more desired compatible-cell-secreted species.

21. The method of any one of the preceding claims, wherein step (c) comprises producing blood progenitor cells and/or HSCs and/or MSCs and/or embryoid bodies and/or RPEs and/or chondrocytes from the one or more iPSCs identified as compatible with the particular subject or particular group of subjects.

22. The method of claim 21, comprising producing the iPSC-derived secretome composition from the produced blood progenitor cells, and/or produced HSCs, and/or produced MSCs, and/or produced embryoid bodies, and/or produced RPEs, and/or produced chondrocytes.

23. The method of any one of the preceding claims, wherein the iPSC-derived secretome composition is a treatment spray, or a treatment cream, or a lotion.

24. The method of any one of claims 1 to 22, wherein the iPSC-derived secretome composition is a treatment injection.

25. A method of manufacturing an induced pluripotent stem cell (iPSC)-derived secretome composition tailored for treatment of a particular subject or particular group of subjects, said method comprising the steps of:
(a) storing, by a processor of a computing device, a database comprising a data entry corresponding to each of a plurality of characterized cells in a physical repository, wherein the characterized cells comprise iPSCs and/or iPSC-derived cells;
(b) receiving, by the processor, a query from a user comprising an identification of a cell type of the particular subject or particular group of subjects;
(c) matching, by the processor, the query to one or more data entries of the database, each of the matching data entries corresponding to each of the plurality of characterized cells having a cell type compatible with the particular subject or particular group of subjects, thereby identifying as compatible with the subject the one or more characterized cells;
(d) retrieving, from a physical repository, compatible cells corresponding to the one or more characterized cells identified as compatible with the particular subject or particular group of subjects; and
(e) producing the iPSC-derived secretome composition using the retrieved compatible cells.

26. The method of claim 25, wherein:
the data entry corresponding to each of the plurality of characterized cells comprises a set of characterized HLA loci corresponding to the cell,
the query comprises a set of queried HLA loci for the particular subject or the particular group of subjects, and
the one or more matched data entries of the database are each representative of one or more characterized compatible cells matching the queried HLA loci.

27. The method of claim 26, wherein the plurality of characterized cells in the physical repository are immortalized.

28. The method of claim 26 or claim 27, wherein the set of characterized HLA loci corresponding to each of the plurality of characterized cells comprises a set of at least 3 HLA loci, wherein the at least 3 HLA loci are HLA-A, HLA-B, and HLA-DRB.

29. The method of claim 26 or claim 27, wherein the set of characterized HLA loci corresponding to each of the plurality of characterized cells comprises a set of at least 9 given loci, wherein the at least 9 given loci are HLA-A, HLA-B, HLA-C, HLA-DRB1, HLA-DRB3, HLA-DRB4, HLA-DRB5, HLA-DQB1, HLA-DPB1.

30. The method of claim 28, wherein the set of characterized HLA loci corresponding to each of the plurality of characterized cells comprises at least 3 given loci selected from the group consisting of HLA-A, HLA-B, HLA-C, HLA-DRB1, HLA-DRB3, HLA-DRB4, HLA-DRB5, HLA-DQB1, and HLA-DPB1.

31. The method of any one of claims 26 to 30, wherein each of the one or more matching data entries of the database exactly match or partially match the set of queried HLA loci for the particular subject or the particular group of subjects.

32. The method of any one of claims 25 to 31, wherein the data entry for each of the plurality of characterized cells further comprises ABO blood type and the query further comprises ABO blood type, and wherein the one or more matching data entries of the database representative of the one or more characterized compatible cells match the queried HLA loci and the queried ABO blood type.

33. The method of any one of claims 25 to 32, wherein the data entry for each of the plurality of characterized cells further comprises RHD blood group and the query further comprises RHD blood group, and wherein the one or more matching data entries of the database representative of the one or more characterized compatible cells match the queried RHD blood group and the queried HLA loci.

34. The method of any one of claims 25 to 33, wherein the queried HLA loci correspond to the particular subject or particular group of subjects in need of an HLA matched iPSC-derived secretome composition.

35. The method of claim 34, wherein the HLA matched iPSC-derived secretome composition is selected from one or more iPSC-derived secretome compositions, each derived from the one or more characterized compatible cells corresponding to each of the one or more data entries of the database that exactly match or partially match the queried HLA loci of the particular subject.

36. The method of any one of claims 25 to 35, wherein one or more of the queried HLA loci is determined by processing and analyzing a biological sample from the particular subject in need of the HLA match.

37. The method of claim 32, wherein the queried ABO blood type, is determined by processing and analyzing a biological sample from the particular subject in need of an ABO match.

38. The method of claim 33, wherein the queried RHD blood group is determined by processing and analyzing a biological sample from the particular subject in need of a RHD blood group match.

39. The method of any one of claims 25 to 38, wherein the physical repository comprises one or more liquid nitrogen storage tanks.

40. The method of any one of claims 25 to 39, comprising producing blood progenitor cells and/or HSCs and/or MSCs and/or RPEs and/or chondrocytes from each of the one or more characterized compatible cells corresponding to the one or more data entries matching the queried HLA loci.

41. The method of any one of claims 25 to 40, further comprising administering the iPSC-derived secretome composition to the particular subject or particular group of subjects.

42. The method of claim 41, wherein the administering step comprises administering the iPSC-derived secretome composition to the particular subject or particular group of subjects for treatment of a known disease, injury, or condition in the particular subject or particular group of subjects, wherein the known disease, injury, or condition is a member selected from the group consisting of lung disease, rheumatic diseases, cardiovascular disease, cancer, arthritis, traumatic brain injury, central nervous system (CNS) injury, and inflammation.

43. The method of any one of claims 25 to 42, wherein the database comprises a data entry corresponding to each of a plurality of iPSC super donor cell lines, wherein the data entry for each super donor cell line comprises a set of characterized HLA loci corresponding to the super donor cell line.

44. The method of claim 43, wherein each of the plurality of iPSC super donor cell lines can be used for treatment of a particular subject or particular group of subjects having matching HLA loci with lower risk of immune rejection by the particular subject or particular group of subjects.

45. The method of claim 43 or claim 44, further comprising determining the set of characterized HLA loci corresponding to each of the plurality of super donor cell lines by processing and analyzing one or more biological samples collected from each of one or more super donor individuals.

46. The method of claim 45, wherein the step of determining the set of characterized HLA loci corresponding to each of the plurality of super donor cell lines comprises identifying a set of at least 3 HLA loci, wherein the at least 3 HLA loci are HLA-A, HLA-B, and HLA-DRB.

47. The method of claim 45, wherein the step of determining the set of characterized HLA loci corresponding to each of the plurality of the super donor cell lines comprises identifying a set of at least 9 HLA loci, wherein the at least 9 HLA loci are HLA-A, HLA-B, HLA-C, HLA-DRB1, HLA-DRB3, HLA-DRB4, HLA-DRB5, HLA-DQB1, and HLA-DPB1.

48. The method of claim 47, wherein the set of characterized HLA loci corresponding to each of the plurality of the super donor cell lines comprises at least 3 HLA loci selected from the group consisting of HLA-A, HLA-B, HLA-C, HLA-DRB1, HLA-DRB3, HLA-DRB4, HLA-DRB5, HLA-DQB1, and HLA-DPB1.

49. The method of any one of claims 43 to 48, wherein the set of characterized HLA loci corresponding to each of the plurality of the super donor cell lines are homozygous for HLA-A, HLA-B, and DRB-1.

50. The method of claim 49, wherein the homozygous set of characterized HLA loci belong to a set of most-common HLA loci for a given population that matches a majority of the given population.

51. The method of any one of claims 43 to 50, wherein the homozygous set of characterized HLA loci comprise homozygous HLA loci in at least 3 major sites wherein the major sites are members selected from the group consisting of HLA-A, HLA-B, HLA-C, HLA-DRB1, HLA-DRB3, HLA-DRB4, HLA-DRB5, HLA-DQB1, and HLA-DPB1.

52. The method of any one of claims 43 to 51, wherein the plurality of iPSC super donor cell lines match at least 70% of the population from which the particular subject originates.

53. The method of any one of claims 43 to 52, wherein the iPSC-derived secretome composition is produced using one of the plurality of iPSC super donor cell lines.

54. The method of claim 53, comprising exposing the iPSC super donor cell line used to produce the iPSC-derived secretome composition to culture media.

55. The method of claim 54, wherein the iPSC-derived secretome composition comprises cells from the iPSC super donor cell line, the culture media, and one or more desired compatible-cell-secreted species.

56. The method of claim 55, wherein the one or more desired compatible-cell-secreted species comprise one or more desired compatible-cell-secreted molecules and/or one or more desired compatible-cell-secreted biological elements.

57. The method of claim 55, wherein the one or more desired compatible-cell-secreted species comprise one or more exosomes and/or one or more microvesicles.

58. The method of any one of claims 43 to 57, comprising producing blood progenitor cells and/or HSCs and/or MSCs and/or RPEs and/or chondrocytes from each of one or more iPSC super donor cell lines identified as compatible with the particular subject or particular group of subjects.

59. The method of any one of claims 25 to 58, wherein the iPSC-derived secretome composition is a treatment spray.

60. The method of any one of claims 25 to 58, wherein the iPSC-derived secretome composition is a treatment lotion or a treatment cream.

61. The method of any one of claims 25 to 60, wherein the iPSC-derived secretome composition comprises one or more proteins listed in Table 1, and/or Table 2, and/or Table 3, and/or Table 4.

62. The method of any one of claims 25 to 61, wherein the iPSC-derived secretome composition is for internal use.

63. The method of claim 62, wherein the iPSC-derived secretome composition is an injection.

64. The method of any one of claims 25 to 61, wherein the iPSC-derived secretome composition is lyophilized.

65. The method of any one of claims 25 to 61, comprising engineering the compatible cells to upregulate production of one or more desired proteins in the iPSC-derived secretome composition.

66. The method of claim 65, wherein the compatible cells are engineered using CRISPR/Cas9 technology.

67. The method of claim 65 or 66, comprising removing and/or replacing and/or editing one or more genes of the compatible cells so as to increase the likelihood of the upregulation of one or more desired proteins in the iPSC-derived secretome composition.

68. A composition of matter comprising an iPSC-derived secretome composition comprising one or more desired compatible-cell-secreted species, wherein the composition is produced by the method of any one of the preceding claims.

69. The composition of claim 68, wherein the iPSC-derived secretome composition is a member selected from the group consisting of a treatment spray, a treatment cream, a treatment lotion, and a treatment injection.

70. The composition of claim 68 or 69, wherein the iPSC-derived secretome composition comprises compatible cells, conditioned culture media, and one or more of the desired compatible-cell-secreted species.

71. The composition of claim 70, wherein the iPSC-derived secretome composition comprises one or more additives.

72. The composition of claim 71, wherein the one or more additives comprises one or more nutrients and/or one or more supplements.

73. The composition of any one of claims 68 to 72, wherein the iPSC-derived secretome composition comprises iPS cells that are derived from a biological sample of a particular subject.

74. The composition of any one of claims 68 to 73, wherein the iPSC-derived secretome composition comprises compatible cells retrieved from a physical repository, wherein the compatible cells are identified as compatible with the particular subject or a particular group of subjects.

75. The composition of claim 74, wherein the compatible cells are identified as compatible with the particular subject or the particular group of subjects using an identification of cell type indicative of compatibility with the particular subject or particular group of subjects, wherein the identification of cell type indicative of compatibility comprises one or more of (i) to (iii): (i) an HLA match, (ii) an ABO blood type match, and (iii) an RHD blood group match having the same HLA loci, and/or ABO blood type, and/or RHD blood group as the.

76. The composition of any one of claims 68 to 75, wherein the iPSC-derived secretome composition comprises one or more compatible-cell-secreted species.

77. The composition of claim 76, wherein the one or more compatible-cell-secreted species are one or more members selected from the group consisting of cytokines, miRNA, siRNA, proteins, organic molecules, inorganic molecules, and biological elements.

78. The composition of any one of claims 68 to 77, wherein the iPSC-derived secretome composition comprises one or more proteins listed in Table 1, and/or Table 2, and/or Table 3, and/or Table 4.

79. The composition of any one of claims 68 to 78, wherein the iPSC-derived secretome composition is formulated internal use.

80. The composition of any one of claim 79, wherein the iPSC derived secretome composition is formulated for use in an injection.

81. The composition of any one of claims 68 to 78, wherein the iPSC-derived secretome composition is lyophilized.

82. The composition of any one of claims 68 to 81, wherein the iPSC-derived secretome composition comprises engineered compatible cells.

83. The composition of claim 82, wherein the engineered compatible cells are modified to upregulate and/or downregulate production of one or more desired proteins in the iPSC-derived secretome composition.

84. The composition of claim 82 or claim 83, wherein the engineered compatible cells are modified using CRISPR/Cas9 technology.

85. A method of storing an induced pluripotent stem cell (iPSC)-derived secretome composition tailored for treatment of a particular subject or particular group of subjects, said method comprising the steps of:
(a) identifying, by a processor of a computing device, as compatible with the particular subject or particular group of subjects, one or more iPSC-derived secretome compositions derived using compatible cells corresponding to the one or more iPSCs and/or iPSC-derived cells identified as compatible with the particular subject or particular group of subjects;
(b) labelling, by a processor of a computing device, the one or more iPSC-derived secretome compositions with a label, wherein the label comprises information relating to the iPSCs and/or iPSC-derived cells, and a classification of the iPSC and/or IPSC-derived cells the iPSC-derived secretome composition is derived from; and
(c) storing, by a processor of a computing device, a database comprising a data entry corresponding to each label in a physical repository.

86. The method of claim 85, wherein the label is a physical label and/or a digital label.

87. The method of claim 85 or claim 86, wherein the label comprises information relating to one or more of (i) to (iii) as follows:
(i) the iPSCs and/or iPSC-derived cells the iPSC-derived secretome composition is derived from;
(ii) one or more HLA loci, and/or ABO blood type, and/or RHD blood group compatible with the labeled iPSC-derived secretome composition; and
(iii) one or more other iPSC-derived secretome compositions stored in the physical repository that are compatible with the particular subject or particular group of subjects, wherein the HLA loci, and/or ABO blood type, and/or RHD blood group of the one or more other iPSC-derived secretome compositions are identical to or match the HLA loci, and/or ABO blood type, and/or RHD blood group of the iPSCs and/or iPSC-derived cells of (i).

88. A method of retrieving one or more produced, labeled and stored iPSC-derived secretome compositions derived using iPSCs and/or iPSC-derived cells, said method comprising the steps of:
(a) identifying, by a processor of a computing device, as compatible with a particular subject or particular group of subjects, one or more iPSC-derived secretome compositions derived using one or more iPSCs and/or iPSC-derived cells identified as compatible with the particular subject or particular group of subjects;
(b) retrieving from a physical repository the one or more compatible iPSC-derived secretome compositions corresponding to the one or more iPSCs and/or iPSC-derived cells identified as compatible with the particular subject or particular group of subjects; and
(c) updating, by a processor of a computing device, a database comprising data entries corresponding to the particular subject or particular group of subjects.

89. The method of claim 88, wherein the retrieved one or more iPSC-derived secretome compositions is administered as treatment to the subject.

90. The method of claim 89, wherein the treatment is a spray.

91. The method of claim 89, wherein the treatment is a cream and/or lotion.

92. The method of claim 89, wherein the treatment is an injection.

93. A method of administering an iPSC-derived secretome composition tailored for treatment of a particular subject or particular group of subjects, said method comprising the steps of:
(a) identifying the particular subject or particular group of subjects as having a deficiency in one or more substances;
(b) identifying, as compatible with the particular subject or particular group of subjects, one or more iPSCs and/or one or more iPSC-derived cells;
(c) retrieving compatible cells corresponding to the one or more iPSCs and/or one or more iPSC-derived cells identified as compatible with the particular subject or particular group of subjects;
(d) producing the iPSC-derived secretome composition using the retrieved compatible cells, wherein the iPSC-derived secretome composition comprises the one or more substances deficient in the particular subject or the particular group of subjects; and
(e) administering to the particular subject or particular group of subjects the iPSC-derived secretome composition.

94. The method of claim 93, wherein the one or more substances comprise one or more cell-secreted molecules and/or cell-secreted biological elements.

95. The method of claim 93 or claim 94, wherein the iPSC-derived secretome composition comprises the one or more cell-secreted substances identified to be deficient in the particular subject or the particular group of subjects.

96. The method of any one of claims 93 to 95, wherein step (d) comprises extracting the secretomes of the retrieved compatible cells.

97. The method of any one of claims 93 to 96, wherein step (c) comprises obtaining the compatible cells from a physical repository.

98. The method of any one of claims 93 to 97, wherein the compatible cells are one or more members selected from the group consisting of iPSCs, MSCs, RPEs, chondrocytes, embryoid bodies, HSCs, and blood progenitor cells.

99. The method of any one of claims 93 to 98, wherein step (c) comprises retrieving the compatible cells using a processor-based query from a user, wherein the query comprises an identification of a cell type indicative of compatibility with the particular subject or particular group of subjects.

100. The method of claim 99, wherein the identification of cell type indicative of compatibility with the particular subject or particular group of subjects comprises one or more of (i) to (iii): (i) an HLA match, (ii) an ABO blood type match, and (iii) an RHD blood group match.

101. The method of any one of claims 93 to 100, wherein step (b) comprises identifying, one or more stored and labeled iPSC-derived secretome compositions within the physical repository derived using one or more iPSCs and/or one or more iPSC-derived cells identified as compatible with the particular subject or group of subjects.

102. The method of any one of claims 93 to 101, wherein step (c) comprises retrieving, the one or more identified iPSC-derived secretome compositions corresponding to the one or more iPSCs and/or one or more iPSC-derived cells identified as compatible with the particular subject or particular group of subjects.

103. The method of any one of claims 93 to 102, wherein step (d) comprises producing a lyophilized iPSC-derived secretome composition.

104. The method of any one of claims 93 to 103, wherein the iPSC-derived secretome composition is administered as treatment to the particular subject or particular group of subjects.

105. The method of claim 104, wherein the treatment is a spray.

106. The method of claim 104, wherein the treatment is a cream and/or lotion.

107. The method any one of claims 93 to 106, wherein the iPSC-derived secretome composition comprises one or more proteins listed in Table 1, and/or Table 2, and/or Table 3, and/or Table 4.

108. The method any one of claims 93 to 107, wherein the iPSC-derived secretome composition is for internal use.

109. The method of claim 108, wherein the iPSC derived secretome composition is an injection.

110. The method of any one of claims 93 to 109, wherein the iPSC-derived secretome composition is lyophilized.

111. The method of any one of claims 93 to 110, comprising engineering the compatible cells to upregulate and/or downregulate production of one or more desired proteins in the iPSC-derived secretome composition.

112. The method of claim 111, wherein the compatible cells are engineered using CRISPR/Cas9 technology.

113. The method of claim 111 or claim 112, comprising removing and/or replacing and/or editing one or more genes of the compatible cells so as to increase the likelihood of the upregulation and/or downregulation of one or more desired proteins in the iPSC-derived secretome composition.

114. The method of any one of claims 93 to 113, wherein the iPSC-derived secretome composition comprises exosomes.

115. The method of any one of claims 93 to 114, wherein the iPSC-derived secretome composition comprises microvesicles.

116. The method of claim 114, wherein the exosomes comprise proteins, and/or siRNAs, and/or miRNAs.

117. The method of claim 115, wherein the microvesicles comprise proteins, and/or siRNAs, and/or miRNAs.

118. The method of any one of claims 93 to 117, wherein the iPSC-derived secretome composition comprises one or more compatible cell types.

119. A method of treating a condition in a subject, the method comprising:
identifying, as compatible with the subject, an iPSC-derived secretome composition; and
administering the iPSC-derived secretome composition to the subject.

120. The method of claim 119, wherein the iPSC-derived secretome composition comprises one or more proteins listed in Table 1, and/or Table 2, and/or Table 3, and/or Table 4.

121. The method of claim 119 or claim 120, wherein the step of identifying the compatible iPSC-derived secretome composition comprises the steps of:
determining HLA loci, and/or ABO blood type, and/or RHD blood group associated with one or more iPSCs and/or one or more iPSC-derived cells from which the iPSC-derived secretome composition is derived; and
matching, by a processor of a computing device, the determined HLA loci, and/or ABO blood type, and/or RHD blood group of the iPSC-derived secretome composition with the HLA loci, and/or ABO blood type, and/or RHD blood group of the subject, wherein a match is an exact match or a partial match.
